SEQUENCE LISTING (37 sequences)

SEQ ID NO: 1 - DNA, 33 bp, Artificial Sequence
  Description of Artificial Sequence: Synthetic primer
gactacgtccacgccctggtcacctattttaat

SEQ ID NO: 2 - DNA, 24 bp, Artificial Sequence
  Description of Artificial Sequence: Synthetic primer
aaggaatccggagcagatgagaag

SEQ ID NO: 3 - DNA, 23 bp, Artificial Sequence
  Description of Artificial Sequence: Synthetic primer
ctgtttgaatgtgtgccaggttg

SEQ ID NO: 4 - DNA, 25 bp, Artificial Sequence
  Description of Artificial Sequence: Synthetic primer
tgaatgtgtgccaggttgaatggag

SEQ ID NO: 5 - DNA, 33 bp, Artificial Sequence
  Description of Artificial Sequence: Synthetic primer
ggtctcgcacatttttggcatttggcttcatgg

SEQ ID NO: 6 - DNA, 28 bp, Artificial Sequence
  Description of Artificial Sequence: Synthetic primer
ggcataggagtcgaagtaataatctctc

SEQ ID NO: 7 - protein, 385 aa, Homo sapiens
MESLASDGFKLKEVSSVNSPPSQPPQPVVPAKPVQCVHHVSTQPSCPGRGKMSKLLNPEEMTSRDYYFDSYAHFGIHEEMLKDEVRTLTYRNSMYHNKHVFKDKVVLDVGSGTGILSMFAAKAGAKKVFGIECSSISDYSEKIIKANHLDNIITIFKGKVEEVELPVEKVDIIISEWMGYCLFYESMLNTVIFARDKWLKPGGLMFPDRAALYVVAIEDRQYKDFKIHWWENVYGFDMTCIRDVAMKEPLVDIVDPKQVVTNACLIKEVDIYTVKTEELSFTSAFCLQIQRNDYVHALVTYFNIEFTKCHKKMGFSTAPDAPYTHWKQTVFYLEDYLTVRRGEEIYGTISMKPNAKNVRDLDFTVDLDFKGQLCETSVSNDYKMR

SEQ ID NO: 8 - DNA, 1995 bp, Homo sapiens
atttctgcaccagggaggcttgctgtttgaatgtgtgccaggttgaatggagtctctggcttcagatggattcaagctgaaagaggtttcttctgtgaacagccccccctcccagcccccccagcccgtcgtccctgctaagcccgtgcaatgcgtccatcatgtgtccactcaacccagctgcccaggacggggcaagatgtccaagctgctgaacccagaggagatgacctcgagagattattacttcgactcctatgcccactttgggatccacgaggaaatgctgaaggatgaggtgcggactctcacttaccggaactccatgtaccacaacaagcacgtgttcaaggacaaagtggtactggatgtggggagtggtactgggatcctttccatgttcgctgccaaggcaggggccaagaaggtgtttgggatcgaatgctccagtatttctgactactcagagaagatcattaaggccaaccacttggacaacatcatcaccatatttaagggtaaagtggaagaggtggagctgcctgtggagaaggtggacatcatcatcagcgagtggatgggctactgtctgttctatgagtccatgctcaacacggtgatctttgccagggacaagtggctgaaacctggagggcttatgtttccagaccgggcagctttgtacgtggtagcgattgaagacagacagtacaaggacttcaaaatccactggtgggagaatgtctatggctttgacatgacctgcatccgggacgtggccatgaaggagcctctagtggacatcgtggatccaaagcaagtggtgaccaatgcctgtttgataaaggaggtggacatttacacagtgaagacggaagagctatcgttcacatctgcattctgcctgcagatacagcgcaacgactacgtccacgccctggtcacctattttaatattgaatttaccaagtgccacaagaaaatggggttttccacagcccctgatgctccctacacccactggaagcagaccgtcttctacttggaagattacctcactgtccggaggggggaggaaatctacgggaccatatccatgaagccaaatgccaaaaatgtgcgagacctcgatttcacagtagacttggattttaagggacagctgtgtgaaacatctgtatctaatgactacaaaatgcgttagcacacgtgggaagctgcagagagcaacgagaaaaggaactctcacctcgatctgccgtgccgtcccaaagaataccgtttgcaggactacacacttgaaaaccagagttttcaactctgccttgaagattggtgaactccccagggctcccgtgggctctgccactggacagaaggcctccagctcctccgctctgccctggtagcccttcacgaaggctttgtgttgccaacaaagagcgacctggcgtgctgtggctgggccccgagggtggaaacgtattcgcgtctccccgtctcctccttaactgtgactctccgggtcttctgagttttgcatgctgcgggtgtctaggacagattgcttccactagaacctggagacatagcatctttgatagcataagccagattatctgtgtgtgcggtggtgtgcgtgtgcgtgcatgtgtgaatgtgagcagcatagttgatatttacccacaaacacctgtatatgcgtgcatatacaaccaagtgggtagacctaggtgttctctcagaggggtgtgtgtgtgtgtgcgtgcgcgtgtgcctagaatatatattactctcagaggagattctgttgcttttgaataggaatttgttttgtgattagttcgccccttccccaccccttaccagatgttaagcagctatgaaacattctctgtactagttctggtctccttttgactggactgtggctctgaaccttgagcatagtaccacggactccgtgggcgctcaataaacacacatgagaacaaa

SEQ ID NO: 9 - DNA, 19 bp, Artificial Sequence
  Description of Artificial Sequence: Synthetic primer
cgagacctcgatttcacag

SEQ ID NO: 10 - DNA, 20 bp, Artificial Sequence
  Description of Artificial Sequence: Synthetic primer
cttggcagcgaacatggaaa

SEQ ID NO: 11 - DNA, 24 bp, Artificial Sequence
  Description of Artificial Sequence: Synthetic primer
caccagtggattttgaagtccttg

SEQ ID NO: 12 - DNA, 21 bp, Artificial Sequence
  Description of Artificial Sequence: Synthetic primer
catccagtaccactttgtcct

SEQ ID NO: 13 - DNA, 22 bp, Artificial Sequence
  Description of Artificial Sequence: Synthetic primer
ctggaaacataagccctccagg

SEQ ID NO: 14 - DNA, 20 bp, Artificial Sequence
  Description of Artificial Sequence: Synthetic primer
ctctggtataaggcggtccc

SEQ ID NO: 15 - DNA, 22 bp, Artificial Sequence
  Description of Artificial Sequence: Synthetic primer
agctgaccctgaagttcatctg

SEQ ID NO: 16 - DNA, 22 bp, Artificial Sequence
  Description of Artificial Sequence: Synthetic primer
tctggcaccacaccttctacaa

SEQ ID NO: 17 - skipped

SEQ ID NO: 18 - DNA, 24 bp, Artificial Sequence
  Description of Artificial Sequence: Synthetic primer
ctgcttgtcggccatgatatagac

SEQ ID NO: 19 - DNA, 21 bp, Artificial Sequence
  Description of Artificial Sequence: Synthetic primer
gctcatcccattagccaaggt

SEQ ID NO: 20 - protein, 394 aa, Homo sapiens
MGMKHSSRCLLLRRKMAENAAESTEVNSPPSQPPQPVVPAKPVQCVHHVSTQPSCPGRGKMSKLLNPEEMTSRDYYFDSYAHFGIHEEMLKDEVRTLTYRNSMYHNKHVFKDKVVLDVGSGTGILSMFAAKAGAKKVFGIECSSISDYSEKIIKANHLDNIITIFKGKVEEVELPVEKVDIIISEWMGYCLFYESMLNTVIFARDKWLKPGGLMFPDRAALYVVAIEDRQYKDFKIHWWENVYGFDMTCIRDVAMKEPLVDIVDPKQVVTNACLIKEVDIYTVKTEELSFTSAFCLQIQRNDYVHALVTYFNIEFTKCHKKMGFSTAPDAPYTHWKQTVFYLEDYLTVRRGEEIYGTISMKPNAKNVRDLDFTVDLDFKGQLCETSVSNDYKMR

SEQ ID NO: 21 - DNA, 2417 bp, Homo sapiens
gtgttgcttcgcccagcggatcggcagaagttgagaggagttggcggctgcctccggccggccggactttgcgagcagcctggagaggatccgcgaccgccgccgccgccgccgcggaggcttcggggctgcttccctcgagcttagcccgcagcgcgggtggagaggggcggggagggggtcgggggcacgagaagaacttgaaaccgtgtgaaggaatccggagcagatgagaagggaggaaaataaaagaaagtggagactgcagaacagactccgctgtggctgactgtgccggccgacgctccagctgaggggctgggttggatttttttttttctcccatcctctcgctctctcttttaaagcgacaccagctctctctcctcctctactatctcggtatcaccaaacccttgccggctcttatgggcatgaaacactcctcccgctgcctgctcctgaggaggaaaatggcggagaacgcggccgagagcaccgaggtgaacagccccccctcccagcccccccagcccgtcgtccctgctaagcccgtgcaatgcgtccatcatgtgtccactcaacccagctgcccaggacggggcaagatgtccaagctgctgaacccagaggagatgacctcgagagattattacttcgactcctatgcccactttgggatccacgaggaaatgctgaaggatgaggtgcggactctcacttaccggaactccatgtaccacaacaagcacgtgttcaaggacaaagtggtactggatgtggggagtggtactgggatcctttccatgttcgctgccaaggcaggggccaagaaggtgtttgggatcgaatgctccagtatttctgactactcagagaagatcattaaggccaaccacttggacaacatcatcaccatatttaagggtaaagtggaagaggtggagctgcctgtggagaaggtggacatcatcatcagcgagtggatgggctactgtctgttctatgagtccatgctcaacacggtgatctttgccagggacaagtggctgaaacctggagggcttatgtttccagaccgggcagctttgtacgtggtagcgattgaagacagacagtacaaggacttcaaaatccactggtgggagaatgtctatggctttgacatgacctgcatccgggacgtggccatgaaggagcctctagtggacatcgtggatccaaagcaagtggtgaccaatgcctgtttgataaaggaggtggacatttacacagtgaagacggaagagctatcgttcacatctgcattctgcctgcagatacagcgcaacgactacgtccacgccctggtcacctattttaatattgaatttaccaagtgccacaagaaaatggggttttccacagcccctgatgctccctacacccactggaagcagaccgtcttctacttggaagattacctcactgtccggaggggggaggaaatctacgggaccatatccatgaagccaaatgccaaaaatgtgcgagacctcgatttcacagtagacttggattttaagggacagctgtgtgaaacatctgtatctaatgactacaaaatgcgttagcacacgtgggaagctgcagagagcaacgagaaaaggaactctcacctcgatctgccgtgccgtcccaaagaataccgtttgcaggactacacacttgaaaaccagagttttcaactctgccttgaagattggtgaactccccagggctcccgtgggctctgccactggacagaaggcctccagctcctccgctctgccctggtagcccttcacgaaggctttgtgttgccaacaaagagcgacctggcgtgctgtggctgggccccgagggtggaaacgtattcgcgtctccccgtctcctccttaactgtgactctccgggtcttctgagttttgcatgctgcgggtgtctaggacagattgcttccactagaacctggagacatagcatctttgatagcataagccagattatctgtgtgtgcggtggtgtgcgtgtgcgtgcatgtgtgaatgtgagcagcatagttgatatttacccacaaacacctgtatatgcgtgcatatacaaccaagtgggtagacctaggtgttctctcagaggggtgtgtgtgtgtgtgcgtgcgcgtgtgcctagaatatatattactctcagaggagattctgttgcttttgaataggaatttgttttgtgattagttcgccccttccccaccccttaccagatgttaagcagctatgaaacattctctgtactagttctggtctccttttgactggactgtggctctgaaccttgagcatagtaccacggactccgtgggcgctcaataaacacacatgagaacaaaaaaaaaaaaaaaa

SEQ ID NO: 22 - protein, 385 aa, Homo sapiens; identical to SEQ ID NO: 7

SEQ ID NO: 23 - DNA, 1995 bp, Homo sapiens; identical to SEQ ID NO: 8

SEQ ID NO: 24 - protein, 392 aa, Homo sapiens
MKHSSRCLLLRRKMAENAAESTEVNSPPSQPPQPVVPAKPVQCVHHVSTQPSCPGRGKMSKLLNPEEMTSRDYYFDSYAHFGIHEEMLKDEVRTLTYRNSMYHNKHVFKDKVVLDVGSGTGILSMFAAKAGAKKVFGIECSSISDYSEKIIKANHLDNIITIFKGKVEEVELPVEKVDIIISEWMGYCLFYESMLNTVIFARDKWLKPGGLMFPDRAALYVVAIEDRQYKDFKIHWWENVYGFDMTCIRDVAMKEPLVDIVDPKQVVTNACLIKEVDIYTVKTEELSFTSAFCLQIQRNDYVHALVTYFNIEFTKCHKKMGFSTAPDAPYTHWKQTVFYLEDYLTVRRGEEIYGTISMKPNAKNVRDLDFTVDLDFKGQLCETSVSNDYKMR

SEQ ID NO: 25 - protein, 348 aa, Homo sapiens
MKHSSRCLLLRRKMAENAAESTEVNSPPSQPPQPVVPAKPVQCVHHVSTQPSCPGRGKMSKLLNPEEMTSRDYYFDSYAHFGIHEEMLKDEVRTLTYRNSMYHNKHVFKDKVVLDVGSGTGILSMFAAKAGAKKVFGIECSSISDYSEKIIKANHLDNIITIFKGKVEEVELPVEKVDIIISEWMGYCLFYESMLNTVIFARDKWLKPGGLMFPDRAALYVVAIEDRQYKDFKIHWWENVYGFDMTCIRDVAMKEPLVDIVDPKQVVTNACLIKEVDIYTVKTEELSFTSAFCLQIQRNDYVHALVTYFNIEFTKCHKKMGFSTAPDAPYTHWKQTVFYLEDYLTVRR

SEQ ID NO: 26 - DNA, 46 bp, Artificial Sequence
  Description of Artificial Sequence: Synthetic oligonucleotide
accacttggacaacatcatcacgagtgatgatgttgtccaagtggt

SEQ ID NO: 27 - DNA, 46 bp, Artificial Sequence
  Description of Artificial Sequence: Synthetic oligonucleotide
agctttgtacgtggtagcgatcgagatcgctaccacgtacaaagct

SEQ ID NO: 28 - DNA, 46 bp, Artificial Sequence
  Description of Artificial Sequence: Synthetic oligonucleotide
ggaagcagaccgtcttctactcgagagtagaagacggtctgcttcc

SEQ ID NO: 29 - DNA, 33 bp, Artificial Sequence
  Description of Artificial Sequence: Synthetic primer
gactacgtccacgccctggtcacctattttatt

SEQ ID NO: 30 - DNA, 22 bp, Artificial Sequence
  Description of Artificial Sequence: Synthetic primer
cttctccttaatgtcacgcacg

SEQ ID NO: 31 - DNA, 137 bp, Artificial Sequence
  Description of Artificial Sequence: Synthetic polynucleotide
tctgcagtcgacggtaccgcgggcccgactacgtccacgccctggtcacctattttaatattgaatttaccaagtgccacaggaaaatggggttttccacagcccctgatgctccctacacccactggaagcagacc

SEQ ID NO: 32 - DNA, 134 bp, Artificial Sequence
  Description of Artificial Sequence: Synthetic polynucleotide
gcagaccgtcttctacttggaagattacctcactgtccgaagggggaggaaatctacgggaccatatccatgaagccaaatgccaaaaatgtgcgagaccgggatcccgcgactctagataattctaccgggta

SEQ ID NO: 33 - DNA, 115 bp, Artificial Sequence
  Description of Artificial Sequence: Synthetic polynucleotide
tgtgccccgaggttttccaggggggggggggggggggccctaaaaaaatttgaaaccgtgggaaggaatccggcgccgaaggaaaggggggaaaaaaaaagaaaggggggactgc

SEQ ID NO: 34 - DNA, 116 bp, Artificial Sequence
  Description of Artificial Sequence: Synthetic polynucleotide
actgcagaacagaatcccctgggggcgtctgtgccggccgaccctccaggggggggggggggttgggtttttttttttctcccatcctctcgctctctcttttaaagcgacaccag

SEQ ID NO: 35 - DNA, 115 bp, Artificial Sequence
  Description of Artificial Sequence: Synthetic polynucleotide
ccagctctctctcctcctctactatctcggtatcaccaaacccttgccggctcttatgggcatgaaacactcctcccgctgcctgctcctgaggaggaaaatgcggagaacgcgg

SEQ ID NO: 36 - DNA, 116 bp, Artificial Sequence
  Description of Artificial Sequence: Synthetic polynucleotide
aacgcggccgagagcaccgaggtgaacagccccccctcccagcccccccagcccgtcgtccctgctaagcccgtgcaatgcgtccatcatgtgtccactcaacccagctgcccagg

SEQ ID NO: 37 - DNA, 127 bp, Artificial Sequence
  Description of Artificial Sequence: Synthetic polynucleotide
  modified_base (2)..(2): a, c, t, g, unknown or other
  modified_base (11)..(11): a, c, t, g, unknown or other
  modified_base (23)..(23): a, c, t, g, unknown or other
  modified_base (25)..(25): a, c, t, g, unknown or other
  modified_base (27)..(27): a, c, t, g, unknown or other
  modified_base (30)..(30): a, c, t, g, unknown or other
tnttttttccnccagggaggctngntnttngaatgtgtgccaggttgaatagactctttggcttcagatggattcaagctgaaagtggtttcttctgtgaacagccccccctcccagcccccccagc

(19) United States
(12) Patent Application Publication
(10) Pub. No.: US 2016/0168647 A1
(43) Pub. Date: Jun. 16, 2016

(54) CHARACTERIZATION OF PRE-CANCER BIOMARKER FOR PROGNOSTIC SCREEN

(71) Applicant: Worcester Polytechnic Institute, Worcester, MA (US)

(72) Inventors: Sarah Hernandez, Worcester, MA (US); Tanja Dominko, Jefferson, MA (US)

(21) Appl. No.: 14/966,539

(22) Filed: Dec. 11, 2015

Related U.S. Application Data
(60) Provisional application No. 62/090,591, filed on Dec. 11, 2014.

(51) Int. Cl. C12Q 1/68; G01N 33/574; A61B 18/02; C12N 9/10

(52) U.S. Cl. CPC C12Q 1/6886; C12N 9/1007; G01N 33/57496; A61B 18/02; C12Q 2600/158; C12Q 2600/16; G01N 2333/91011

(57) ABSTRACT

The invention features compositions and methods for a pre-cancer prognostic screen.

### RELATED APPLICATIONS

This application claims the benefit of priority under 35 U.S.C. §119(e) to U.S. Provisional Application No: 62/090,591, filed Dec. 11, 2014, which is incorporated herein by reference in its entirety.

### STATEMENT OF RIGHTS TO INVENTIONS MADE UNDER FEDERALLY SPONSORED RESEARCH

This work was supported by the National Institutes of Health under grant number R01GM85456 and by the National Science Foundation under grant number DGE 1144804. The Government has certain rights in this invention.

### FIELD OF THE INVENTION

This invention relates generally to the field of cancer.

### BACKGROUND OF THE INVENTION

Cancer is one of the most prevalent diseases, accounting for 25% of all deaths in the United States. As such, medicine has shifted from reactive to proactive treatment options. Colonoscopies alone have reduced morality from colorectal cancer by 53%. As medical technology advances, preventative screens are becoming less invasive and more widespread as research reveals biomarkers that can be used to identify cancer-related changes. However, prior to the invention described herein, there were no biomarkers widely used in cancer screens prior to tumor formation.

### SUMMARY OF THE INVENTION

The invention is based, at least in part, on the surprising discovery that a messenger ribonucleic acid (mRNA) variant of protein arginine methyltransferase 8 (PRMT8) is upregulated in cells that resemble “pre-cancerous” cells. As described in detail below, PRMT8 is used as a biomarker in a simple, inexpensive test to identify “pre-cancerous” cells.

Described herein are kits for detecting the expression of PRMT8 comprising a PRMT8-specific primer. For example, the PRMT8-specific primer comprises one of the following nucleic acid sequences or pairs of nucleic acid sequences:

TABLE 1

| | Fwd primer (5′ to 3′) | Rev primer (5′ to 3′) |
|---|---|---|
| PRMT8 | GACTACGTCCACGCCCTGGTCACCT | GGTCTCGCACATTTTTGGCATTTGGCTTCATG |
| | ATTTTAAT (SEQ ID NO: 1) | G (SEQ ID NO: 5) |
| PRMT8 v1 | AAGGAATCCGGAGCAGATGAGAAG | GGCATAGGAGTCGAAGTAATAATCTCTC |
| | (SEQ ID NO: 2) | (SEQ ID NO: 6) |
| PRMT8 v2 | CTGTTTGAATGTGTGCCAGGTTG | GGCATAGGAGTCGAAGTAATAATCTCTC |
| | (SEQ ID NO: 3) | (SEQ ID NO: 6) |
| PRMT8 v2 | TGAATGTGTGCCAGGTTGAATGGA | GGCATAGGAGTCGAAGTAATAATCTCTC |
| nested | G (SEQ ID NO: 4) | (SEQ ID NO: 6) |

The invention also provides an isolated PRMT8 polypeptide variant, e.g., a synthetic isolated PRMT8 polypeptide variant. For example, provided is a PRMT8 polypeptide variant comprising the following amino acid sequence, (GenBank Accession Number NP_001243465 (NP_001243465.1), incorporated herein by reference):

(SEQ ID NO: 7)
MESLASDGFKLKEVSSVNSPPSQPPQPVVPAKPVQCVHHVSTQPSCPGRG
KMSKLLNPEEMTSRDYYFDSYAHFGIHEEMLKDEVRTLTYRNSMYHNKHV
FKDKVVLDVGSGTGILSMFAAKAGAKKVFGIECSSISDYSEKIIKANHLD
NIITIFKGKVEEVELPVEKVDIIISEWMGYCLFYESMLNTVIFARDKWLK
PGGLMFPDRAALYVVAIEDRQYKDFKIHWWENVYGFDMTCIRDVAMKEPL
VDIVDPKQVVTNACLIKEVDIYTVKTEELSFTSAFCLQIQRNDYVHALVT
YFNIEFTKCHKKMGFSTAPDAPYTHWKQTVFYLEDYLTVRRGEEIYGTIS
MKPNAKNVRDLDFTVDLDFKGQLCETSVSNDYKMR

Described herein is also an isolated nucleotide sequence encoding the isolated PRMT8 polypeptide variant, e.g., a synthetic PRMT8 nucleic acid sequence and/or PRMT8 complementary deoxyribonucleic acid (cDNA). In some cases, the isolated nucleic acid sequence is immobilized on a solid support. In one aspect, the isolated nucleic acid sequence is linked to a detectable label. Exemplary detectable labels include a fluorescent label, a luminescent label, a chemiluminescent label, a radiolabel, a SYBR Green label, and a Cy3-label.

For example, a PRMT8 transcript variant 2 comprises the mRNA sequence set forth in GenBank Accession Number NM001256536 (NM_001256536.1), incorporated herein by reference:

| (SEQ ID NO: 8) | |
|---|---|
| 1 | atttctgcac cagggaggct tgctgtttga atgtgtgcca ggttgaatgg agtctctggc |
| 61 | ttcagatgga ttcaagctga aagaggtttc ttctgtgaac agccccccct cccagccccc |
| 121 | ccagcccgtc gtccctgcta agcccgtgca atgcgtccat catgtgtcca ctcaacccag |
| 181 | ctgcccagga cggggcaaga tgtccaagct gctgaaccca gaggagatga cctcgagaga |
| 241 | ttattacttc gactcctatg cccactttgg gatccacgag gaaatgctga aggatgaggt |
| 301 | gcggactctc acttaccgga actccatgta ccacaacaag cacgtgttca aggacaaagt |
| 361 | ggtactggat gtggggagtg gtactgggat cctttccatg ttcgctgcca aggcaggggc |
| 421 | caagaaggtg tttgggatcg aatgctccag tatttctgac tactcagaga agatcattaa |
| 481 | ggccaaccac ttggacaaca tcatcaccat atttaagggt aaagtggaag aggtggagct |
| 541 | gcctgtggag aaggtggaca tcatcatcag cgagtggatg ggctactgtc tgttctatga |
| 601 | gtccatgctc aacacggtga tctttgccag ggacaagtgg ctgaaacctg gagggcttat |
| 661 | gtttccagac cgggcagctt tgtacgtggt agcgattgaa gacagacagt acaaggactt |
| 721 | caaaatccac tggtgggaga atgtctatgg ctttgacatg acctgcatcc gggacgtggc |
| 781 | catgaaggag cctctagtgg acatcgtgga tccaaagcaa gtggtgacca atgcctgttt |
| 841 | gataaaggag gtggacattt acacagtgaa gacggaagag ctatcgttca catctgcatt |
| 901 | ctgcctgcag atacagcgca acgactacgt ccacgccctg gtcacctatt ttaatattga |
| 961 | atttaccaag tgccacaaga aaatggggtt ttccacagcc cctgatgctc cctacaccca |
| 1021 | ctggaagcag accgtcttct acttggaaga ttacctcact gtccggaggg gggaggaaat |
| 1081 | ctacgggacc atatccatga agccaaatgc caaaaatgtg cgagacctcg atttcacagt |
| 1141 | agacttggat tttaagggac agctgtgtga aacatctgta tctaatgact acaaaatgcg |
| 1201 | ttagcacacg tgggaagctg cagagagcaa cgagaaaagg aactctcacc tcgatctgcc |
| 1261 | gtgccgtccc aaagaatacc gtttgcagga ctacacactt gaaaaccaga gttttcaact |
| 1321 | ctgccttgaa gattggtgaa ctccccaggg ctcccgtggg ctctgccact ggacagaagg |
| 1381 | cctccagctc ctccgctctg ccctggtagc ccttcacgaa ggctttgtgt tgccaacaaa |
| 1441 | gagcgacctg gcgtgctgtg gctgggcccc gagggtggaa acgtattcgc gtctccccgt |
| 1501 | ctcctcctta actgtgactc tccgggtctt ctgagttttg catgctgcgg gtgtctagga |
| 1561 | cagattgctt ccactagaac ctggagacat agcatctttg atagcataag ccagattatc |
| 1621 | tgtgtgtgcg gtggtgtgcg tgtgcgtgca tgtgtgaatg tgagcagcat agttgatatt |
| 1681 | tacccacaaa cacctgtata tgcgtgcata tacaaccaag tgggtagacc taggtgttct |
| 1741 | ctcagagggg tgtgtgtgtg tgtgcgtgcg cgtgtgccta gaatatatat tactctcaga |
| 1801 | ggagattctg ttgcttttga ataggaattt gttttgtgat tagttcgccc cttccccacc |
| 1861 | ccttaccaga tgttaagcag ctatgaaaca ttctctgtac tagttctggt ctccttttga |
| 1921 | ctggactgtg gctctgaacc ttgagcatag taccacggac tccgtgggcg ctcaataaac |
| 1981 | acacatgaga acaaa |

Described herein are methods of diagnosing pre-cancer comprising obtaining a test sample from a subject having or at risk of having cancer; determining a level of PRMT8 mRNA in the sample; comparing the level of PRMT8 mRNA in the test sample to a level of PRMT8 in a tissue-matched normal control; identifying an elevated level of PRMT8 mRNA in the test sample compared to the level of PRMT8 mRNA in the tissue-matched normal control, thereby identifying a pre-cancerous cell and diagnosing pre-cancer. Suitable test samples include blood, stool, urine, and saliva.

Methods of detecting a pre-cancerous or cancerous cell are carried out by obtaining a test sample from a subject having or at risk of having cancer; determining the expression level of PRMT8 in the test sample; comparing the expression level of PRMT8 in the test sample with the expression level of PRMT8 in a reference sample; and detecting a pre-cancerous or cancerous cell if the expression level of PRMT8 in the test sample is elevated as compared to the expression level of PRMT8 in the reference sample. The methods described herein include in vitro methods and in vivo methods.

The subject is preferably a mammal in need of such treatment, e.g., a subject that has been diagnosed with cancer or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

In some cases, the methods further comprise administering a chemotherapeutic agent, radiation therapy, cryotherapy, or hormone therapy, thereby inhibiting tumor cell growth in the subject. Exemplary chemotherapeutic agents include doceaxel, cabazitaxel, mitoxantrone, estramustine, doxorubicin, etoposide, and paclitaxel. In one aspect, the methods also include administering an anti-neoplastic agent, wherein the anti-neoplastic agent comprises radiotherapy, a cell death-inducing agent, or a proteasome inhibitor, thereby inhibiting tumor cell growth in the subject.

Preferably, the test sample includes RNA. Preferably, the expression level of PRMT8 mRNA is determined. For example, reverse transcription polymerase chain reaction (RT-PCR) is utilized to determine a level of PRMT8 mRNA in the test sample. In some cases, the PRMT8 in the test sample comprises a PRMT8 mRNA variant comprising the nucleic acid sequence set forth in SEQ ID NO: 8.

Preferably, the methods also include administering an inhibitor of PRMT8 to the subject, thereby inhibiting tumor cell growth. The inhibitors or antagonists may include but are not limited to nucleic acids, peptides, antibodies, or small molecules that bind to their specified target or the target's natural ligand and modulate the biological activity. For example, suitable inhibitors of PRMT8 include a small molecule inhibitor, RNA interferance (RNAi), an antibody, or any combination thereof.

In one aspect, the antagonist or inhibitor comprises an antibody or fragment thereof, a binding protein, a polypeptide, or any combination thereof. Described herein are anti-PRMT8 antibodies. Suitable anti-PRMT8 antibodies include PA5-11310 (Thermo Scientific, Waltham, Mass.), TA302105 (Origene, Rockville, Md.), and GTX47431 (GeneTex, Inc., Irvine, Calif.), each of which is incorporated herein by reference. However, the skilled artisan could readily identify additional anti-PRMT8 antibodies for use in the methods described herein. In some cases, the anti-PRMT8 antibodies described herein are administered at a concentration of 0.1 μg/ml to 500 mg/ml.

In some cases, the antagonist comprises a small molecule. A small molecule is a compound that is less than 2000 Daltons in mass. The molecular mass of the small molecule is preferably less than 1000 Daltons, more preferably less than 600 Daltons, e.g., the compound is less than 500 Daltons, less than 400 Daltons, less than 300 Daltons, less than 200 Daltons, or less than 100 Daltons.

Small molecules are organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecules can be synthetically engineered to consist of a fragment, or small portion, or a longer amino acid chain to fill a binding pocket of an enzyme. Typically, small molecules are less than one kilodalton.

In some cases, the antagonist comprises a nucleic acid molecule. For example, RNA or deoxyribonucleic acid (DNA) inhibits the expression of PRMT8 polypeptide, thereby inhibiting the activity of PRMT8. In some cases, the nucleic acid comprises small interfering RNA (siRNA), RNA interference (RNAi), messenger RNA (mRNA), small hairpin RNA or short hairpin RNA (shRNA), double stranded ribonucleic acid (dsRNA), antisense RNA, or microRNA, or any portion thereof. Thus, suitable PRMT8 antagonists include PRMT8 siRNA, which is available from, e.g., ThermoFisher Scientific, and incorporated herein by reference. Similarly, suitable PRMT8 antagonists include PRMT8 shRNA, which is available from, e.g., Origene, Rockville, Md., and incorporated herein by reference. However, the skilled artisan could readily identify additional nucleic acids that inhibit/antagonize PRMT8.

The effective amount of the antagonist is from 0.001 mg/kg to 250 mg/kg body weight, e.g., 0.001 mg/kg, 0.05 mg/kg 0.01 mg/kg, 0.05 mg/kg, 1 mg/kg, 5 mg/kg, 10 mg/kg, 25 mg/kg, 50 mg/kg, 75 mg/kg, 100 mg/kg, 125 mg/kg, 150 mg/kg, 175 mg/kg, 200 mg/kg, 225 mg/kg, or 250 mg/kg body weight. Ultimately, the attending physician or veterinarian decides the appropriate amount and dosage regimen.

In some cases, the antagonist or inhibitor is administered at least once per day, at least once per week, or at least once per month. The antagonist is administered for a duration of one day, one week, one month, two months, three months, six months, 9 months, or one year. In some cases, the antagonist is administered daily, e.g., every 24 hours. Or, the antagonist is administered continuously or several times per day, e.g., every 1 hour, every 2 hours, every 3 hours, every 4 hours, every 5 hours, every 6 hours, every 7 hours, every 8 hours, every 9 hours, every 10 hours, every 11 hours, or every 12 hours.

Optionally, the reference sample comprises a tissue-matched normal control sample. Exemplary test samples include a plasma sample, a blood sample, and a tissue sample. Preferably, the reference sample is obtained from a healthy normal control subject.

The methods described herein are useful in treating, delaying the progression of, preventing relapse of or alleviating a symptom of a cancer or other neoplastic or pre-neoplastic condition. For example, the methods described herein are useful in treating hematological malignancies and/or tumors. For example, the methods described herein are useful in treating non-Hodgkin's lymphoma (NHL), acute lymphocytic leukemia (ALL), acute myeloid leukemia (AML), chronic lymphocytic leukemia (CLL), chronic myelogenous leukemia (CML), multiple myeloma (MM), breast cancer, ovarian cancer, head and neck cancer, bladder cancer, melanoma, colorectal cancer, pancreatic cancer, lung cancer, colon cancer, leiomyoma, leiomyosarcoma, glioma, glioblastoma, and so on. Solid tumors include, e.g. , breast tumors, ovarian tumors, lung tumors, pancreatic tumors, prostate tumors, melanoma tumors, colorectal tumors, lung tumors, head and neck tumors, bladder tumors, esophageal tumors, liver tumors, and kidney tumors. As used herein, “hematological cancer” refers to a cancer of the blood, and includes leukemia, lymphoma and myeloma among others. “Leukemia” refers to a cancer of the blood in which too many white blood cells that are ineffective in fighting infection are made, thus crowding out the other parts that make up the blood, such as platelets and red blood cells. It is understood that cases of leukemia are classified as acute or chronic. Certain forms of leukemia include, by way of non-limiting example, acute lymphocytic leukemia (ALL); acute myeloid leukemia (AML); chronic lymphocytic leukemia (CLL); chronic myelogenous leukemia (CML); Myeloproliferative disorder/neoplasm (MPDS); and myelodysplasia syndrome. “Lymphoma” may refer to a Hodgkin' s lymphoma, both indolent and aggressive non-Hodgkin's lymphoma, Burkitt's lymphoma, and follicular lymphoma (small cell and large cell), among others. “Myeloma” may refer to multiple myeloma (MM), giant cell myeloma, heavy-chain myeloma, and light chain or Bence-Jones myeloma.

In some cases, the methods described herein are used in conjunction with one or more agents or a combination of additional agents, e.g., an antineoplastic agent. Suitable agents include current pharmaceutical and/or surgical therapies for an intended application, such as, for example, cancer or pre-cancer. For example, the methods described herein can be used in conjunction with one or more chemotherapeutic or anti-neoplastic agents. In some cases, the additional chemotherapeutic agent is radiotherapy. In some cases, the chemotherapeutic agent is a cell death-inducing agent. In some embodiments, the chemotherapeutic agent is a proteasome inhibitor.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

By “agent” is meant any small compound, antibody, nucleic acid molecule, or polypeptide, or fragments thereof.

By “alteration” is meant a change (increase or decrease) in the expression levels or activity of a gene or polypeptide as detected by standard art-known methods such as those described herein. As used herein, an alteration includes at least a 1% change in expression levels, e.g., at least a 2%, 3%, 4%, 5%, 6%, 7%, 8%, 9%, 10%, 20%, 30%, 40%, 50%, 60%, 70%, 80%, 90%, or 100% change in expression levels. For example, an alteration includes at least a 5%-10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease, e.g., cancer.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

By “binding to” a molecule is meant having a physicochemical affinity for that molecule. By “control” or “reference” is meant a standard of comparison. As used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, β-galactosidase or luciferase). Depending on the method used for detection, the amount and measurement of the change can vary. Determination of statistical significance is within the ability of those skilled in the art, e.g., the number of standard deviations from the mean that constitute a positive result.

“Detect” refers to identifying the presence, absence, or amount of the agent (e.g., a nucleic acid molecule, for example deoxyribonucleic acid (DNA) or ribonucleic acid (RNA)) to be detected.

By “detectable label” is meant a composition that when linked (e.g., joined—directly or indirectly) to a molecule of interest renders the latter detectable, via, for example, spectroscopic, photochemical, biochemical, immunochemical, or chemical means. Direct labeling can occur through bonds or interactions that link the label to the molecule, and indirect labeling can occur through the use of a linker or bridging moiety which is either directly or indirectly labeled. Bridging moieties may amplify a detectable signal. For example, useful labels may include radioactive isotopes, magnetic beads, metallic beads, colloidal particles, fluorescent labeling compounds, electron-dense reagents, enzymes (for example, as commonly used in an enzyme-linked immunosorbent assay (ELISA)), biotin, digoxigenin, or haptens. When the fluorescently labeled molecule is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, p-phthaldehyde and fluorescamine. The molecule can also be detectably labeled using fluorescence emitting metals such as 152 Eu, or others of the lanthanide series. These metals can be attached to the molecule using such metal chelating groups as diethylenetriaminepentacetic acid (DTPA) or ethylenediaminetetraacetic acid (EDTA). The molecule also can be detectably labeled by coupling it to a chemiluminescent compound. The presence of the chemiluminescent-tagged molecule is then determined by detecting the presence of luminescence that arises during the course of chemical reaction. Examples of particularly useful chemiluminescent labeling compounds are luminol, isoluminol, theromatic acridinium ester, imidazole, acridinium salt and oxalate ester.

A “detection step” may use any of a variety of known methods to detect the presence of nucleic acid. The types of detection methods in which probes can be used include Western blots, Southern blots, dot or slot blots, and Northern blots.

As used herein, the term “diagnosing” refers to classifying pathology or a symptom, determining a severity of the pathology (e.g., grade or stage), monitoring pathology progression, forecasting an outcome of pathology, and/or determining prospects of recovery.

By the terms “effective amount” and “therapeutically effective amount” of a formulation or formulation component is meant a sufficient amount of the formulation or component, alone or in a combination, to provide the desired effect. For example, by “an effective amount”is meant an amount of a compound, alone or in a combination, required to ameliorate the symptoms of a disease, e.g., cancer, relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

The term “pre-cancer” is used herein to refer to cells that are not presently cancerous, but are likely to develop into tumor forming cells. “Pre-cancer” may also refer to cells that are cancerous, but have yet to metastasize.

A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “immobilized” or “attached” refers to a probe (e.g., nucleic acid or protein) and a solid support in which the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule to the support and the non-covalent binding of a biotinylated probe to the molecule. Immobilization may also involve a combination of covalent and non-covalent interactions.

By “marker” is meant any protein or polynucleotide having an alteration in expression level or activity that is associated with a disease or disorder, e.g., cancer.

By “modulate” is meant alter (increase or decrease). Such alterations are detected by standard art-known methods such as those described herein.

The term, “normal amount” refers to a normal amount of a complex in an individual known not to be diagnosed with a disease, e.g., cancer. The amount of the molecule can be measured in a test sample and compared to the “normal control level” utilizing techniques such as reference limits, discrimination limits, or risk defining thresholds to define cutoff points and abnormal values (e.g., for cancer). The “normal control level” means the level of one or more proteins (or nucleic acids) or combined protein indices (or combined nucleic acid indices) typically found in a subject known not to be suffering from a disease, e.g., cancer. Such normal control levels and cutoff points may vary based on whether a molecule is used alone or in a formula combining other proteins into an index. Alternatively, the normal control level can be a database of protein patterns from previously tested subjects who did not convert to cancer over a clinically relevant time horizon.

Relative to a control level, the level that is determined may be an increased level. As used herein, the term “increased” with respect to level (e.g., expression level, biological activity level, etc.) refers to any % increase above a control level. The increased level may be at least or about a 1% increase, at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, or at least or about a 95% increase, relative to a control level.

Relative to a control level, the level that is determined may be a decreased level. As used herein, the term “decreased” with respect to level (e.g., expression level, biological activity level, etc.) refers to any % decrease below a control level. The decreased level may be at least or about a 1% decrease, at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, or at least or about a 95% decrease, relative to a control level.

By “neoplasia” is meant a disease or disorder characterized by excess proliferation or reduced apoptosis. Illustrative neoplasms for which the invention can be used include, but are not limited to pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

The term “overexpress” or “overexpression” refers to a situation in which more factor is expressed by a genetically-altered cell than would be, under the same conditions, by a wild type cell. Similarly, if an unaltered cell does not express a factor that it is genetically altered to produce, the term “express” (as distinguished from “overexpress”) is used indicating the factor the wild type cell did not express the factor at all prior to genetic manipulation

The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “sample” as used herein refers to a biological sample obtained for the purpose of evaluation in vitro. Exemplary tissue samples for the methods described herein include tissue samples from pre-cancerous or cancerous tissue. With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). Preferred samples are whole blood, serum, plasma, or urine. A sample can also be a partially purified fraction of a tissue or bodily fluid.

A reference sample can be a “normal” sample, e.g., from a donor not having the disease or condition, fluid or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

A “solid support” describes a strip, a polymer, a bead, or a nanoparticle. The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with a disease (e.g., cancer) is within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

In some cases, a composition of the invention is administered orally or systemically. Other modes of administration include rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

Pharmaceutical compositions may be assembled into kits or pharmaceutical systems for use in arresting cell cycle in rapidly dividing cells, e.g., cancer cells. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit.

Unless specifically stated or obvious from context, as used herein, the term “or” is understood to be inclusive. Unless specifically stated or obvious from context, as used herein, the terms “a”, “an”, and “the” are understood to be singular or plural.

Any compositions or methods provided herein can be combined with one or more of any of the other compositions and methods provided herein.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is a schematic of protein variants of PRMT8 and their localization.

FIG. 2A is a bar chart showing the five most up- and down-regulated chromatin modifiers in extended lifespan (ELS) cells compared to control cells.

FIG. 2B is a photograph of a blot showing the results of RT-PCR of PRMT8 in various cell types compared to actin.

FIG. 2C is a photograph of a western blot of PRMT8 in various cell types compared to actin.

FIG. 3 is a series of photomicrographs showing PRMT8 localization in ELS and control (Ctl) cells.

FIG. 4 is a photograph of a blot showing PRMT8 variant transcript expression by RT-PCR in ELS cells and hESCs.

FIG. 5A is a line graph showing cumulative population doublings of control cells under normal conditions, with a scramble negative control, and with three separate PRMT8 short hairpin ribonucleic acids (shRNAs).

FIG. 5B is a series of photomicrographs showing immunocytochemistry of cells from each treatment group at passage 10.

FIG. 5C is a series of photomicrographs showing immunocytochemistry of cells from each treatment group at passage 13, 15 days after being transferred to ELS culture conditions.

FIG. 6 is a schematic showing derivation of the induced regeneration competent (iRC) phenotype. Supplementation with the growth factor FGF2 under reduced oxygen over 7 day culture period leads to increased plasticity of adult human fibroblasts characterized by a pro-regenerative, non-tumorigenic phenotype.

FIG. 7A-FIG. 7B is a series of bar charts showing the effect of culture conditions on expression of chromatin modification enzymes. Target genes were analyzed using a qRT-PCR Chromatin Modification Enzyme Array (SA Biosciences) (n=1). FIG. 7A shows fold change in expression of the top 5 most up- and down-regulated chromatin modifiers as normalized to the housekeeping gene RPL13A. FIG. 7B shows fold change in expression in members of the PRMT family normalized to the housekeeping gene RPL13A.

FIG. 8A-FIG. 8D is a series of photographs of blots and a bar chart showing the effect of culture conditions on PRMT8 expression. FIG. 8A shows PRMT8 transcript expression in control human dermal fibroblasts (CRL-2352), iRC cells, and hESCs compared to expression in mouse brain by RT-PCR. Actin was used as a loading control. FIG. 8B shows PRMT8 protein expression in control human dermal fibroblasts (CRL-2352), iRC cells, and hESCs compared to expression of purified GST-tagged PRMT8. Actin was used as a loading control. 10 μg of total protein were loaded in each lane except for GST-PRMT8 lanes (0.25 μg and 0.5 μg, respectively). Antibody dilutions are as follows: PRMT8—1:200, actin—1:5000, HRP anti-Rb—1:10,000. FIG. 8C shows densitometric representation of protein levels normalized to actin from three separate experiments. *All treatments were significantly different from each other: control compared to iRC, p=0.0002; control compared to hESCs, p =0.002; iRC compared to hESCs, p=0.02. FIG. 8D shows PRMT8 transcript expression in various control human dermal fibroblast lines (CRL-2352; CRL-2097; CT-1005) compared to the same lines grown under iRC conditions by RT-PCR. Actin was used as a loading control.

FIG. 9A and FIG. 9B is a series of graphs showing PRMT8 transcript sequence. FIG. 9A shows a graphic representation of PRMT8 and amplicon location. Boxes represent the 10 exons of PRMT8 where the dashed line in exon 1 represents the lengths of the alternative 5′ exons that differentiate variant 1 from variant 2. The grey line that spans a portion of exons 8-10 represents the amplicon that was sequenced. FIG. 9B shows PRMT8 cDNA from iRC cells was cloned intopLVX at Smal (CCCGGG), and sequenced. The grey line in the sequencing data represents the grey amplicon in FIG. 9A.

FIG. 10A and FIG. 10B are a series of schematics showing graphic representation of PRMT8 transcript variants, protein isoforms, and localization differences. FIG. 10A shows the genomic alignment of PRMT8 transcript variants 1 and 2. Numbers represent million base pairs, dashed lines represent introns, and solid vertical lines represent exons. FIG. 10B shows both PRMT8 mRNA variants are expressed from the PMRT8 gene on chromosome 12. mRNA variant 1 has 3 alternative translation start sites, responsible for protein isoforms 1-3. mRNA variant 2 is transcribed from an alternative 5′ exon and is responsible for translation of isoform 4. Isoform 1 harbors an N-terminal myristoylation motif, represented by the red coil, conferring plasma membrane localization. Isoforms 2 and 3 are truncated at the N-terminus and display nuclear localization. Isoform 4 is a variant that has not been explored experimentally. Conserved PRMT core regions are represented in grey, methyltransferase domains are represented in black, and the conserved THW loop is represented in blue. The unique portion of the protein sequence for isoform 4 is represented in orange.

FIG. 11A-FIG. 11D are a series of graphs and a photograph of a blot showing PRMT8 variant expression. FIG. 11A shows the sequence for 5′ RACE of hESCs-PRMT8 compared to sequences in the NCBI database. Asterisks represent base pairs mismatched from the NCBI database. Dashed line represents beginning of alignment with database sequences, which begins with the 172nd nucleotide. Solid lines represent exon-exon junctions. FIG. 11B shows the sequence for 5′ RACE of iRC-PRMT8 compared to sequences in the NCBI database. Asterisks represent base pairs mismatched from the NCBI database, and “N” represents sequencing misreads. Solid lines represent exon-exon junctions. FIG. 11C shows graphic representation of 5′ RACE data from hESCs and iRC cells compared to variant 1 and variant 2. Solid horizontal lines represent mRNA alignments between treatments and boxes represent exons. Numbers delineate base pairs. FIG. 11D shows variant specific PRMT8 transcript expression in control cells, iRC cells, and hESCs using RT-PCR. Actin was used as a loading control.

FIG. 12A and FIG. 12B are a series of photomicrographs and a photograph of a blot showing demonstration of knockdown specificity. FIG. 12A shows GFP reporter fluorescence of U87MG glioblastomas on day 2 post-transduction. Scale bars are 200 m. FIG. 12B shows transcript expression for PRMT8 variant 2 is compared to PRMT1 in all treatment groups using RT-PCR. Actin and GFP were used as loading controls. Cells were harvested 2 days post-transduction.

FIG. 13A and FIG. 13B is a line graph and a series of photomicrographs demonstrating the effect of PRMT8 on human dermal fibroblast growth and longevity. FIG. 13A shows three independent replicates were performed and cumulative population doublings were measured and averaged for cells in all treatment groups. Error bars represent standard deviation. FIG. 13B shows GFP reporter fluorescence on day 6 (left) and day 14 (right).

FIG. 14A and FIG. 14B is a line graph and a series of hotomicrographs demonstrating the effect of PRMT8 on glioblastoma growth and longevity. FIG. 14A shows that three independent replicates were performed and cumulative population doublings were measured and averaged for cells in all treatment groups. Error bars represent standard deviation. Measurements for cells in the PRMT8 shRNA treatment group were terminated after day 6 due to complete cell loss. FIG. 14B shows GFP reporter fluorescence on day 1 (left) and day 6 (right).

### DETAILED DESCRIPTION

The invention is based, at least in part, on the surprising discovery that an mRNA variant of PRMT8 is upregulated in cells that resemble “pre-cancer.” As described in detail below, PRMT8 is used as a biomarker in a simple, inexpensive test to identify “pre-cancerous” cells. Specifically, as described herein, an mRNA variant of PRMT8 was identified in cells with a prolonged life span and acquired regeneration potential, but without the ability to form tumors. The marker appears before the cells become turmorigenic and can be used for detection of a pre-cancerous state.

Cancer is one of the most prevalent diseases worldwide, accounting for 25% of all deaths in the United States (Siege et al., 2012 Cancer statistics, 2:10-29). As such, medicine has shifted from reactive to proactive therapies. Colonoscopies alone have reduced morality from colorectal cancer by 53% (Zauber et al., 2012 New England Journal of Medicine, 366:687-696). As medical technology advances, preventative screenings are becoming less invasive and more widespread as research reveals biomarkers that can be used to identify cancer-related changes. However, prior to the invention described herein, there were no biomarkers widely used in cancer screens prior to tumor formation. As such, described herein is a prognostic test that intervenes before patients develop cancer by screening for biomarkers of pre-cancerous biological changes with a qualitative diagnostic screening device that detects a biomarker associated with pre-cancerous cells.

Finite cellular proliferative lifespan and onset of irreversible growth arrest, termed “senescence”, has long been recognized in differentiated eukaryotic cells (Kyo et al., 2008 Cancer Science, 99: 1528-1538). Molecular mechanisms that regulate this terminal arrest of the cell cycle, however, can be deregulated, leading to uncontrolled cell proliferation in cancer cells or continuous self-renewal in pluripotent stem cells; both cell types becoming neoplastic in parallel. Six biological capabilities have been detailed during the evolution of healthy cells to a neoplastic state. Of the 6 canonical hallmarks of cancer (resisting cell death, sustaining proliferative signaling, evading growth suppressors, activating invasion and metastasis, enabling replicative immortality, and inducing angiogenesis), four are associated with increased cellular lifespan (Hanahan, D. and Weinberg, R. A. 2000 Cell, 100: 57-70; Hanahan, D. and Weinberg, R. A. 2011 Cell, 144: 646-674). Investigating processes that control lifespan enables progression toward identification of mechanisms that control the switch between normal cell division and neoplastic proliferation.

Methylation is one of the most widely studied and diverse post-translational modifications (PTMs). Methyl groups can be added to the side chains of various amino acids, such as proline, lysine, histidine, and arginine (Lee et al., 2005 Endocrine reviews, 26: 147-170). In particular, arginine methylation can influence biological processes such as transcriptional permissiveness, cellular differentiation, and telomere length and stability (Lee et al., 2005 Endocrine reviews, 26: 147-170; Wang et al., 2001 Science Signaling, 293: 853; Peterson, C. L. and Laniel, M.A. 2004 Current Biology, 14: R546-R551; Yu et al., 2006 Genes & Development, 20: 3249-3254; Iberg et al., 2008 Journal of Biological Chemistry, 283: 3006-3010; Mitchell et al., 2009 Molecular and Cellular Biology, 29: 4918-4934; Tee et al., 2010 Genes & Development, 24: 2772-2777). Many biological processes regulated by arginine methylation are well-described, but prior to the invention described herein limited knowledge existed about how PRMTs themselves are regulated. However, aberrant expression of protein arginine methyltransferase (PRMT) family members has been associated with cardiovascular and pulmonary diseases, as well as various types of cancers including lung, bladder, colon, and breast cancers (Yoshimatsu et al., 2011 International Journal of Cancer, 128: 562-573; Zakrzewicz et al., International Journal of Molecular Sciences, 13: 12383-12400; Mathioudaki et al., 2008 British Journal of Cancer, 99: 2094-2099; Goulet et al., 2007 Journal of Biological Chemistry, 282: 33009-33021).

Protein Arginine methyltransferase 8 (PRMT8)

Arginine methyltransferases have remained grossly understudied given their critical functional roles and variant-specific functions in cancer biology. As described herein, evaluation of PRMT variant expression and regulation reveals critical physiological and pathophysiological mechanisms and leads to therapeutic developments.

Prior to the invention described herein, the function of the protein arginine methyltransferase 8 enzyme was largely uncertain. PRMT8 is a protein that is encoded by the PRMT8 gene in humans. Arginine methylation is a widespread posttranslational modification mediated by arginine methyltransferases, such as PRMT8. Arginine methylation is involved in a number of cellular processes, including DNA repair, RNA transcription, signal transduction, and protein compartmentalization. PRMT8 is a membrane-associated arginine methyltransferase that can both catalyze the formation of omega-N monomethylarginine (MMA) and asymmetrical dimethylarginine (aDMA). For example, PRMT8 binds and dimethylates Ewing sarcoma breakpoint region 1 (EWS) protein. A variety of biological roles for PRMT family members are being uncovered indicating potential regulatory mechanisms for arginine methylation in cellular senescence.

PRMT8 was first identified because of sequence similarity with PRMT1 (Lee et al., 2005 Journal of Biological Chemistry, 280: 32890-32896, incorporated herein by reference), and phylogenetic analysis revealed it to be a paralogue of PRMT1 in vertebrates (Hung, C. M. and Li, C. 2004 Gene, 340: 179-187; Lin et al., 2013 PLOS ONE, 8: e55221). PRMT1 is ubiquitously expressed and is found in both nuclei and cytoplasm (Sayegh et al., 2007 Journal of Biological Chemistry, 282: 36444-36453; Frankel et al., Journal of Biological Chemistry, 277: 3537-3543; Herrmann et al., 2005 Journal of Biological Chemistry, 280: 38005-38010). Although members of the PRMT family are all highly homologous, PRMT8 and PRMT1 are most similar with 83% sequence identity, differing only at the N-terminus, where PRMT8 contains 76 additional amino acids (Lee et al., 2005 Journal of Biological Chemistry, 280: 32890-32896; Sayegh et al., 2007 Journal of Biological Chemistry, 282: 36444-36453; Kousaka et al., 2009 Neuroscience, 163: 1146-1157). Northern blot analysis demonstrated that full-length PRMT8 transcript expression was found largely in brain tissue (Lee et al., 2005 Journal of Biological Chemistry, 280: 32890-32896; Sayegh et al., 2007 Journal of Biological Chemistry, 282: 36444-36453; Taneda et al., 2007 Brain Research, 1155: 1-9). However, analysis of PRMT8 in a non-mammalian vertebrate system found ubiquitous expression during embryonic development, whereas expression only became restricted to brain tissue after neural development (Lin et al., 2013 PLOS ONE, 8: e55221).

PRMT8 has three described isoforms with unique N-termini translated from differing inframe start codons. Early characterization of full length PRMT8 (isoform 1) revealed a glycine residue at the N-terminus modified by a myristoylation motif (Lee et al., 2005 Journal of Biological Chemistry, 280: 32890-32896; Sayegh et al., 2007 Journal of Biological Chemistry, 282: 36444-36453, each of which is incorporated herein by reference). Myristoylation is the addition of a hydrophobic moiety that results in sequestration of modified proteins to the plasma membrane (Lee et al., 2005 Journal of Biological Chemistry, 280: 32890-32896; Sayegh et al., 2007 Journal of Biological Chemistry, 282: 36444-36453). However, overexpressed PRMT8 translated from the second (isoform 2) and third (isoform 3) in-frame start codons displays nuclear localization (Kousaka et al., 2009 Neuroscience, 163: 1146-1157, incorporated herein by reference). In mice, endogenous PRMT8 localizes to nuclei (Kousaka et al., 2009 Neuroscience, 163: 1146-1157). Previous studies of PRMT8 utilized overexpression of the full-length isoform, which guided the consensus that the endogenous isoform is the full-length product and that expression is restricted to brain tissue. If PRMT8 is one of the truncated nuclear isoforms and is expressed more widely than initially reported, it challenges the existing paradigm and it suggests that PRMT8, like other PRMT family members, may have a role in critical cellular processes through chromatin modification or regulation of protein-protein interactions. However, prior to the invention described herein, the expression or function of PRMT8 in human cells was not examined.

An exemplary human PRMT8 amino acid sequence (PRMT8 isoform 1) is set forth below (SEQ ID NO: 20; GenBank Accession No. NP_062828, Version NP_062828.3 (GI:74099699), incorporated herein by reference):

| | |
|---|---|
| 1 | mgmkhssrcl llrrkmaena aestevnspp sqppqpvvpa kpvqcvhhvs tqpscpgrgk |
| 61 | mskllnpeem tsrdyyfdsy ahfgiheeml kdevrtltyr nsmyhnkhvf kdkvvldvgs |
| 121 | gtgilsmfaa kagakkvfgi ecssisdyse kiikanhldn iitifkgkve evelpvekvd |
| 181 | iiisewmgyc lfyesmlntv ifardkwlkp gglmfpdraa lyvvaiedrq ykdfkihwwe |
| 241 | nvygfdmtci rdvamkeplv divdpkqvvt naclikevdi ytvkteelsf tsafclqiqr |
| 301 | ndyvhalvty fnieftkchk kmgfstapda pythwkqtvf yledyltvrr geeiygtism |
| 361 | kpnaknvrdl dftvdldfkg qlcetsysnd ykmr |

An exemplary human PRMT8 nuclic acid sequence (PRMT8, transcript variant 1, mRNA) is set forth below (SEQ ID NO: 21; GenBank Accession No. NM_019854, Version NM_019854.4 (GI:374858038), incorporated herein by reference):

| | |
|---|---|
| 1 | gtgttgcttc gcccagcgga tcggcagaag ttgagaggag ttggcggctg cctccggccg |
| 61 | gccggacttt gcgagcagcc tggagaggat ccgcgaccgc cgccgccgcc gccgcggagg |
| 121 | cttcggggct gcttccctcg agcttagccc gcagcgcggg tggagagggg cggggagggg |
| 181 | gtcgggggca cgagaagaac ttgaaaccgt gtgaaggaat ccggagcaga tgagaaggga |
| 241 | ggaaaataaa agaaagtgga gactgcagaa cagactccgc tgtggctgac tgtgccggcc |
| 301 | gacgctccag ctgaggggct gggttggatt tttttttttc tcccatcctc tcgctctctc |
| 361 | ttttaaagcg acaccagctc tctctcctcc tctactatct cggtatcacc aaacccttgc |
| 421 | cggctcttat gggcatgaaa cactcctccc gctgcctgct cctgaggagg aaaatggcgg |
| 481 | agaacgcggc cgagagcacc gaggtgaaca gccccccctc ccagcccccc cagcccgtcg |
| 541 | tccctgctaa gcccgtgcaa tgcgtccatc atgtgtccac tcaacccagc tgcccaggac |
| 601 | ggggcaagat gtccaagctg ctgaacccag aggagatgac ctcgagagat tattacttcg |
| 661 | actcctatgc ccactttggg atccacgagg aaatgctgaa ggatgaggtg cggactctca |
| 721 | cttaccggaa ctccatgtac cacaacaagc acgtgttcaa ggacaaagtg gtactggatg |
| 781 | tggggagtgg tactgggatc ctttccatgt tcgctgccaa ggcaggggcc aagaaggtgt |
| 841 | ttgggatcga atgctccagt atttctgact actcagagaa gatcattaag gccaaccact |
| 901 | tggacaacat catcaccata tttaagggta aagtggaaga ggtggagctg cctgtggaga |
| 961 | aggtggacat catcatcagc gagtggatgg gctactgtct gttctatgag tccatgctca |
| 1021 | acacggtgat ctttgccagg gacaagtggc tgaaacctgg agggcttatg tttccagacc |
| 1081 | gggcagcttt gtacgtggta gcgattgaag acagacagta caaggacttc aaaatccact |
| 1141 | ggtgggagaa tgtctatggc tttgacatga cctgcatccg ggacgtggcc atgaaggagc |
| 1201 | ctctagtgga catcgtggat ccaaagcaag tggtgaccaa tgcctgtttg ataaaggagg |
| 1261 | tggacattta cacagtgaag acggaagagc tatcgttcac atctgcattc tgcctgcaga |
| 1321 | tacagcgcaa cgactacgtc cacgccctgg tcacctattt taatattgaa tttaccaagt |
| 1381 | gccacaagaa aatggggttt tccacagccc ctgatgctcc ctacacccac tggaagcaga |
| 1441 | ccgtcttcta cttggaagat tacctcactg tccggagggg ggaggaaatc tacgggacca |
| 1501 | tatccatgaa gccaaatgcc aaaaatgtgc gagacctcga tttcacagta gacttggatt |
| 1561 | ttaagggaca gctgtgtgaa acatctgtat ctaatgacta caaaatgcgt tagcacacgt |
| 1621 | gggaagctgc agagagcaac gagaaaagga actctcacct cgatctgccg tgccgtccca |
| 1681 | aagaataccg tttgcaggac tacacacttg aaaaccagag ttttcaactc tgccttgaag |
| 1741 | attggtgaac tccccagggc tcccgtgggc tctgccactg gacagaaggc ctccagctcc |
| 1801 | tccgctctgc cctggtagcc cttcacgaag gctttgtgtt gccaacaaag agcgacctgg |
| 1861 | cgtgctgtgg ctgggccccg agggtggaaa cgtattcgcg tctccccgtc tcctccttaa |
| 1921 | ctgtgactct ccgggtcttc tgagttttgc atgctgcggg tgtctaggac agattgcttc |
| 1981 | cactagaacc tggagacata gcatctttga tagcataagc cagattatct gtgtgtgcgg |
| 2041 | tggtgtgcgt gtgcgtgcat gtgtgaatgt gagcagcata gttgatattt acccacaaac |
| 2101 | acctgtatat gcgtgcatat acaaccaagt gggtagacct aggtgttctc tcagaggggt |
| 2161 | gtgtgtgtgt gtgcgtgcgc gtgtgcctag aatatatatt actctcagag gagattctgt |
| 2221 | tgcttttgaa taggaatttg ttttgtgatt agttcgcccc ttccccaccc cttaccagat |
| 2281 | gttaagcagc tatgaaacat tctctgtact agttctggtc tccttttgac tggactgtgg |
| 2341 | ctctgaacct tgagcatagt accacggact ccgtgggcgc tcaataaaca cacatgagaa |
| 2401 | caaaaaaaaa aaaaaaa |

An exemplary human PRMT8 amino acid sequence (PRMT8 isoform 4) is set forth below (SEQ ID NO: 22; GenBank Accession No. NP_001243465, Version NP_001243465.1 (GI:374858040), incorporated herein by reference):

| | |
|---|---|
| 1 | meslasdgfk lkevssvnsp psqppqpvvp akpvqcvhhv stqpscpgrg kmskllnpee |
| 61 | mtsrdyyfds yahfgiheem lkdevrtlty rnsmyhnkhv fkdkvvldvg sgtgilsmfa |
| 121 | akagakkvfg iecssisdys ekiikanhld niitifkgkv eevelpvekv diiisewmgy |
| 181 | clfyesmlnt vifardkwlk pgglmfpdra alyvvaiedr qykdfkihww envygfdmtc |
| 241 | irdvamkepl vdivdpkqvv tnaclikevd iytvkteels ftsafclqiq rndyvhalvt |
| 301 | yfnieftkch kkmgfstapd apythwkqtv fyledyltvr rgeeiygtis mkpnaknvrd |
| 361 | ldftvdldfk gqlcetsVsn dykmr |

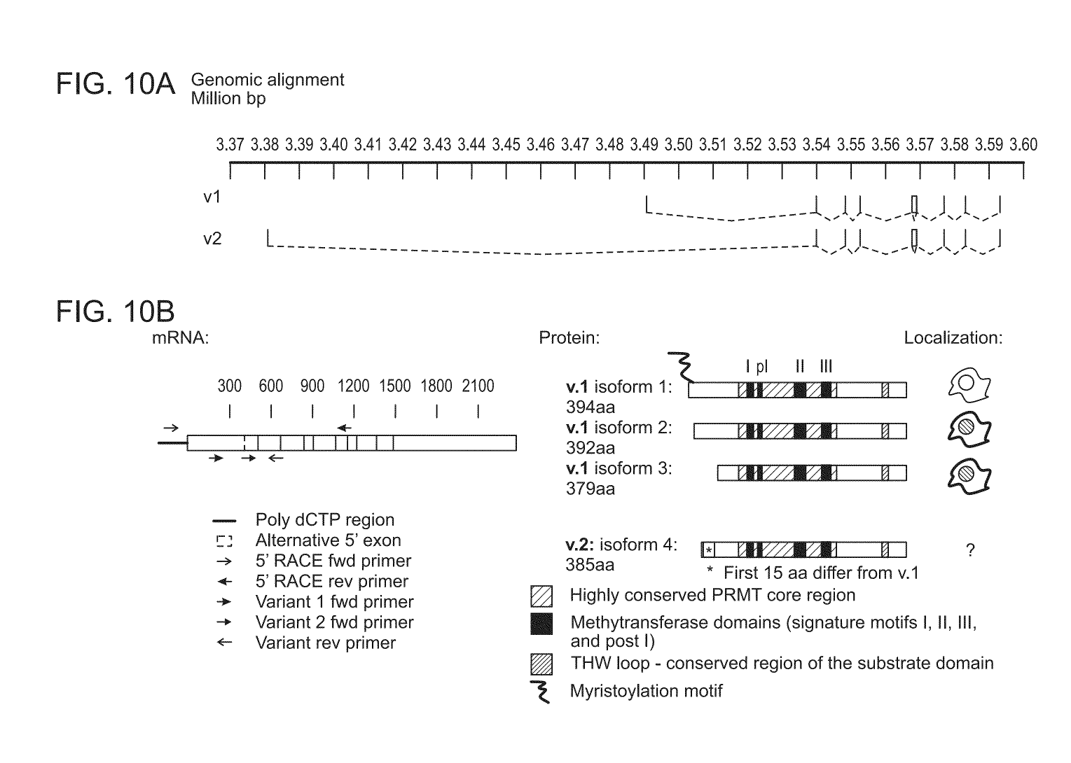

Although SEQ ID NO: 22 is provided as isoform 2 in the National Center for Biotechnology Information (NCBI) database, this sequence is provided as isoform 4 in FIG. 10. Isoforms 1-3 are translated from mRNA variant 1, while isoform 4 is translated from mRNA variant 2.

An exemplary human PRMT8 nuclic acid sequence (PRMT8, transcript variant 2, mRNA) is set forth below (SEQ ID NO: 23; GenBank Accession No. NM_001256536 Version NM_001256536.1 (GI:374858039), incorporated herein by reference):

| | |
|---|---|
| 1 | atttctgcac cagggaggct tgctgtttga atgtgtgcca ggttgaatgg agtctctggc |
| 61 | ttcagatgga ttcaagctga aagaggtttc ttctgtgaac agccccccct cccagccccc |
| 121 | ccagcccgtc gtccctgcta agcccgtgca atgcgtccat catgtgtcca ctcaacccag |
| 181 | ctgcccagga cggggcaaga tgtccaagct gctgaaccca gaggagatga cctcgagaga |
| 241 | ttattacttc gactcctatg cccactttgg gatccacgag gaaatgctga aggatgaggt |
| 301 | gcggactctc acttaccgga actccatgta ccacaacaag cacgtgttca aggacaaagt |
| 361 | ggtactggat gtggggagtg gtactgggat cctttccatg ttcgctgcca aggcaggggc |
| 421 | caagaaggtg tttgggatcg aatgctccag tatttctgac tactcagaga agatcattaa |
| 481 | ggccaaccac ttggacaaca tcatcaccat atttaagggt aaagtggaag aggtggagct |
| 541 | gcctgtggag aaggtggaca tcatcatcag cgagtggatg ggctactgtc tgttctatga |
| 601 | gtccatgctc aacacggtga tctttgccag ggacaagtgg ctgaaacctg gagggcttat |
| 661 | gtttccagac cgggcagctt tgtacgtggt agcgattgaa gacagacagt acaaggactt |
| 721 | caaaatccac tggtgggaga atgtctatgg ctttgacatg acctgcatcc gggacgtggc |
| 781 | catgaaggag cctctagtgg acatcgtgga tccaaagcaa gtggtgacca atgcctgttt |
| 841 | gataaaggag gtggacattt acacagtgaa gacggaagag ctatcgttca catctgcatt |
| 901 | ctgcctgcag atacagcgca acgactacgt ccacgccctg gtcacctatt ttaatattga |
| 961 | atttaccaag tgccacaaga aaatggggtt ttccacagcc cctgatgctc cctacaccca |
| 1021 | ctggaagcag accgtcttct acttggaaga ttacctcact gtccggaggg gggaggaaat |
| 1081 | ctacgggacc atatccatga agccaaatgc caaaaatgtg cgagacctcg atttcacagt |
| 1141 | agacttggat tttaagggac agctgtgtga aacatctgta tctaatgact acaaaatgcg |
| 1201 | ttagcacacg tgggaagctg cagagagcaa cgagaaaagg aactctcacc tcgatctgcc |
| 1261 | gtgccgtccc aaagaatacc gtttgcagga ctacacactt gaaaaccaga gttttcaact |
| 1321 | ctgccttgaa gattggtgaa ctccccaggg ctcccgtggg ctctgccact ggacagaagg |
| 1381 | cctccagctc ctccgctctg ccctggtagc ccttcacgaa ggctttgtgt tgccaacaaa |
| 1441 | gagcgacctg gcgtgctgtg gctgggcccc gagggtggaa acgtattcgc gtctccccgt |
| 1501 | ctcctcctta actgtgactc tccgggtctt ctgagttttg catgctgcgg gtgtctagga |
| 1561 | cagattgctt ccactagaac ctggagacat agcatctttg atagcataag ccagattatc |
| 1621 | tgtgtgtgcg gtggtgtgcg tgtgcgtgca tgtgtgaatg tgagcagcat agttgatatt |
| 1681 | tacccacaaa cacctgtata tgcgtgcata tacaaccaag tgggtagacc taggtgttct |
| 1741 | ctcagagggg tgtgtgtgtg tgtgcgtgcg cgtgtgccta gaatatatat tactctcaga |
| 1801 | ggagattctg ttgcttttga ataggaattt gttttgtgat tagttcgccc cttccccacc |
| 1861 | ccttaccaga tgttaagcag ctatgaaaca ttctctgtac tagttctggt ctccttttga |
| 1921 | ctggactgtg gctctgaacc ttgagcatag taccacggac tccgtgggcg ctcaataaac |
| 1981 | acacatgaga acaaa |

An exemplary human PRMT8 amino acid sequence (PRMT8 isoform 2) is set forth below (SEQ ID NO: 24):

MKHSSRCLLLRRKMAENAAESTEVNSPPSQPPQPVVPAKPVQCVHHVSTQ
PSCPGRGKMSKLLNPEEMTSRDYYFDSYAHFGIHEEMLKDEVRTLTYRNS
MYHNKHVFKDKVVLDVGSGTGILSMFAAKAGAKKVFGIECSSISDYSEKI
IKANHLDNIITIFKGKVEEVELPVEKVDIIISEWMGYCLFYESMLNTVIF
ARDKWLKPGGLMFPDRAALYVVAIEDRQYKDFKIHWWENVYGFDMTCIRD
VAMKEPLVDIVDPKQVVTNACLIKEVDIYTVKTEELSFTSAFCLQIQRND
YVHALVTYFNIEFTKCHKKMGFSTAPDAPYTHWKQTVFYLEDYLTVRRGE
EIYGTISMKPNAKNVRDLDFTVDLDFKGQLCETSVSNDYKMR

PRMT8 isoform 2 is identical to PRMT8 isoform 1; however, PRMT8 isoform 2 is truncated by 2 amino acids at the N-terminus.

An exemplary human PRMT8 amino acid sequence (PRMT8 isoform 3) is set forth below (SEQ ID NO: 25):

MKHSSRCLLLRRKMAENAAESTEVNSPPSQPPQPVVPAKPVQCVHHVSTQ
PSCPGRGKMSKLLNPEEMTSRDYYFDSYAHFGIHEEMLKDEVRTLTYRNS
MYHNKHVFKDKVVLDVGSGTGILSMFAAKAGAKKVFGIECSSISDYSEKI
IKANHLDNIITIFKGKVEEVELPVEKVDIIISEWMGYCLFYESMLNTVIF
ARDKWLKPGGLMFPDRAALYVVAIEDRQYKDFKIHWWENVYGFDMTCIRD
VAMKEPLVDIVDPKQVVTNACLIKEVDIYTVKTEELSFTSAFCLQIQRND
YVHALVTYFNIEFTKCHKKMGFSTAPDAPYTHWKQTVFYLEDYLTVRR

PRMT8 isoform 3 is identical to PRMT8 isoform 1; however, PRMT8 isoform 3 is truncated by 15 amino acids at the N-terminus. PRMT8 isoform is essential for cell viability and proliferation

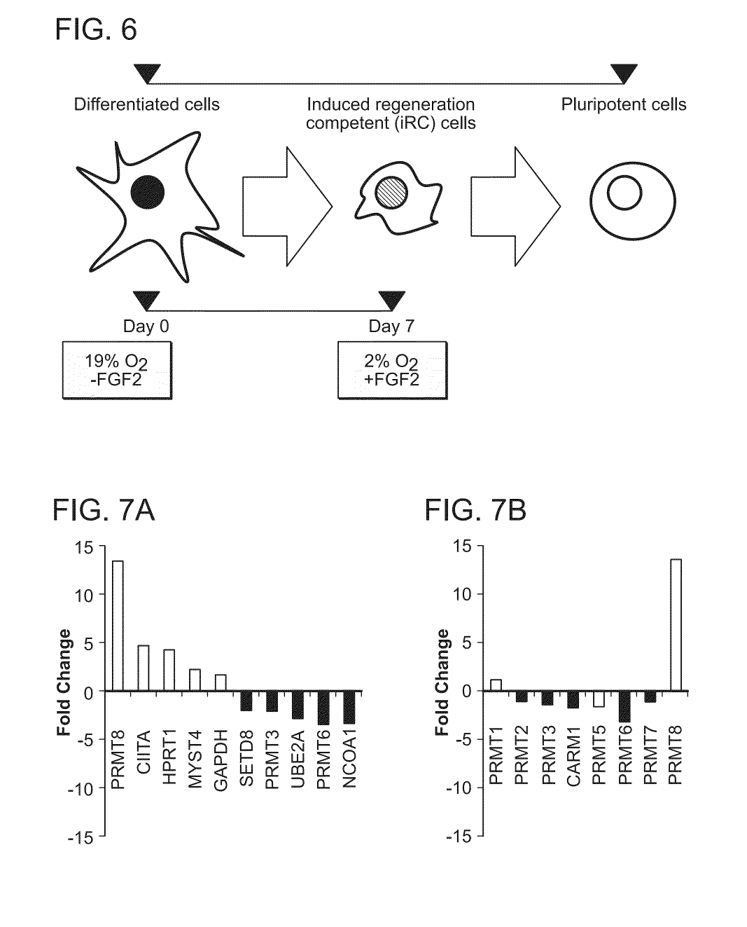

Described herein is the development of a unique, reversible cell phenotype from primary human dermal fibroblasts, termed induced regeneration competent (iRC) cells. iRC cells are derived by exogenous addition of human fibroblast growth factor FGF2 and culture in reduced oxygen concentration (2%) (FIG. 6). Reduction in oxygen concentration has been shown to increase cellular lifespan and to regulate epigenetic changes (Jeltsch, A. 2013 Trends in Biochemical Sciences; Bradley et al., 1978 Journal of cellular physiology, 97: 517-522). iRC cells display increased proliferative lifespan and increased time to cellular senescence while lacking the propensity to form tumors when injected into SCID mice, a capability that is characteristic of immortalized and pluripotent cells (Page, et al., 2009 Cloning and Stem Cells, 11: 417-426; Page et al., 2011 Tissue Engineering Part A, 17: 2629-2640). This unique phenotype allows for the examination of molecular changes that lead to increased cellular lifespan without cancerous permanent self-renewal.

Small molecule inhibitors of enzymes that catalyze PTMs have been approved by the Food and Drug Administration for treatment of human cancers, and arginine methyltransferases are being hailed as the new enzymes to target for personalized cancer therapeutics (Richon, V. M., Moyer, M. P., and Copeland, R. A. (2012) Protein Methyltransferases as Targets for Personalized Cancer; Copeland et al., 2009 Nature Reviews Drug Discovery, 8: 724-732; Ott, P. A. and Adams, S. 2011Immunotherapy, 3: 213-227; Rodriguez-Paredes, M. and Esteller, M. 2011 Nature medicine, 330-339). Prior to the invention described herein, limited evidence about PRMT regulation prevented understanding of biological consequences of corruption in their regulatory pathways. However, this family of enzymes plays a significant role in cell viability and in cancer biology (Yoshimatsu et al., 2011 International Journal of Cancer, 128: 562-573; Zakrzewicz et al., International Journal of Molecular Sciences, 13: 12383-12400; Mathioudaki et al., 2008 British Journal of Cancer, 99: 2094-2099; Goulet et al., 2007 Journal of Biological Chemistry, 282: 33009-33021; Mitchell et al., 2009 Molecular and Cellular Biology, 29: 4918-4934; Leiper, J. and Vallance, P. 1999 Cardiovascular Research, 43: 542-548; Pahlich et al., 2006 Biochimica Et Biophysica Acta-Proteins and Proteomics, 1764: 1890-1903; Wysocka et al., 2006 Frontiers in Bioscience, 11: 344-355; Pal, S. and Sif, S. 2007 Journal of Cellular Physiology, 213: 306-315; Herrmann et al., 2009 Journal of Cell Science, 122: 667-677; Di Lorenzo, A. and Bedford, M. T. 2011 Febs Letters, 585: 2024-2031; Hong et al., 2012 Biogerontology, 13: 329-336; Wang et al., 2008 Molecular and cellular biology, 28: 6262-6277; Yu et al., 2009 Molecular and Cellular Biology, 29: 2982-2996; Bedford, M. T. and Richard, S. 2005 Molecular Cell, 18: 263-272).

PRMT8 specifically has been understudied because of early reports implicating tissue specificity; however, it can no longer be ignored that PRMT8 does in fact have functional relevance outside the brain. An in vivo zebrafish study found that PRMT8 is expressed ubiquitously during early development and is critical for embryonic and neural development, as knockdown of PRMT8 resulted in early developmental defects in all three germ layers and, in many cases, death (Lin et al., 2013 PLOS ONE, 8: e55221). This was the first evidence that PRMT8 plays a critical role in development before becoming localized specifically to mature brain tissue. Described herein is PRMT8 expression in hESCs, the first evidence that PRMT8 may also function in human development. Furthermore, PRMT8 expression is demonstrated in human dermal fibroblast-derived cells, clearly indicating human PRMT8 expression outside of the CNS.

The upregulation of PRMT8 by iRC culture conditions is primarily mediated by culture in reduced oxygen, though it is potentiated by supplementation with fibroblast growth factor 2 (FGF2). Cell culture is routinely performed at atmospheric oxygen levels (between 19% to 20%) even though physiological levels tend to be much lower (ranging from 10% to 0.5%, depending on tissue type) (Dings et al., 1998 Neurosurgery, 43: 1082-1094; Harrison et al., 2002 Blood, 99: 394-394; Pasarica et al., 2009 Diabetes, 58: 718-725; Evans et al., 2006 Journal of investigative dermatology, 126: 2596-2606). As described in detail below, oxygen concentration was reduced in the model system to more closely match the physiological state. The fact that physiological oxygen levels are much lower than what is used for standard cell culture methods, and the fact that brain specifically is a hypoxic tissue (Dings et al., 1998 Neurosurgery, 43: 1082-1094), may be the cause of why PRMT8 has, until now, not been seen widely outside the CNS. It is possible that iRC culture conditions are not inducing PRMT8 expression but, rather, that standard culture conditions are repressing its expression.

The demonstration herein of increased PRMT8 protein expression with reduced oxygen is not the first indication that hypoxic conditions regulate PRMTs. In a study that analyzed PRMT 1-7 in mouse lung tissue, hypoxia was shown to be a regulator of PRMT2 (Yildirim et al., 2006 American Journal of Respiratory Cell and Molecular Biology, 35: 436-443). However, it was noted that PRMT8 was not analyzed alongside other PRMT family members in this study due to its assumed specificity to brain, highlighting the importance of recent literature that has shown PRMT8 to be ubiquitously expressed, at least during development (Lin et al., 2013 PLOS ONE, 8: e55221).

PRMT family members have variant-specific functions in various cancers, which makes them attractive targets for cancer diagnostics and/or therapeutics. For example, specific splice variants of PRMT1 demonstrate distinct activity and substrate specificity and have been correlated to tumor grade in breast cancer (Goulet et al., 2007 Journal of Biological Chemistry, 282: 33009-33021; Scott et al., 1998 Genomics, 48: 330-340; Scorilas et al., 2000 Biochemical and biophysical research communications, 278: 349-359; Mathioudaki et al., 2011 Tumor Biology, 32: 575-582). Nevertheless, the current ability to target these molecules is limited by the lack of understanding regarding expression and regulation of specific PRMT variants and the variant-specific effects they have in cancer cell lines and tumors. Prior to the invention described herein, the mechanism by which a shift from one isoform to another occurs was not known, although this shift is thought to be important for cancer development and progression. Described herein is the identification of an PRMT8 variant expressed in cells grown under iRC culture conditions, conditions that lead to increased cellular lifespan without the capacity to form tumors when injected into SCID mice (Page, et al., 2009 Cloning and Stem Cells, 11: 417-426; Page et al., 2011 Tissue Engineering Part A, 17: 2629-2640). Increased understanding about the role of PRMTs in cancer-related changes (i.e. bypassing the Hayflick limit) in a non-tumorigenic system increases understanding of PRMT regulation while offering molecular tools for development of cancer treatments and diagnostic tests.

The most interesting phenotype observed herein that PRMT8 knockdown leads to a loss of cell proliferation. As described in detail below, fibroblast transductions were performed under control conditions, with the plan to transfer to iRC conditions following selection. However, cells in knockdown treatments failed to recover following transduction, indicating that the small amount of PRMT8 present in control human dermal fibroblasts is necessary for proliferation, regardless of culture conditions. The glioblastoma line U87MG was selected for PRMT8 knockdown due to sole expression of PRMT8 variant 2. Immediate loss of proliferation in this cell type is thought to be the cause of increased sensitivity to transduction compared to primary cell types. These results encourage the continued exploration of PRMT8 as a biomarker and therapeutic target.

While other PRMTs have been robustly linked to cell cycle, this is the first evidence of PRMT8 having a functional role in cell proliferation, suggesting that PRMT8 is more similar to other PRMT family members than initially thought. In human lung fibroblasts, PRMTs 1, 4, and 6 are down-regulated as cells senesce and their expression decreases as p21 increases during senescence (Lim et al., 2008 Journal of biochemistry, 144: 523-529). In osteosarcoma, breast, bladder and lung cancer lines, PRMT1 knockdown results in GO/G1 arrest, a common hallmark of senescent cells (Yoshimatsu et al., 2011 International Journal of Cancer, 128: 562-573; Yu et al., 2009 Molecular and Cellular Biology, 29: 2982-2996; Le Romancer et al., 2008 Molecular cell, 31: 212-221). In mouse embryonic fibroblasts (MEFs), PRMT6 knockdown increases expression of both p53 and p21 (Phalke et al., 2012Nucleic acids research, gks858; Kleinschmidt et al., 2012 PloS one 7, e41446). Because of this, it was hypothesized that the mechanism by which PRMT8 influences cell proliferation is through regulation of cell cycle. However, it remains to be determined exactly which genes and/or proteins are regulated by PRMT8.

Described herein is the upregulation of a specific gene, PRMT8, in cells which resemble “pre-cancer,” which may be used as a biomarker in a simple, inexpensive test as a form of preventative medicine. Also described herein is a prognostic test with pre-cancerous screening capabilities based on up-regulation of this gene.

The screen described herein takes advantage of biological samples obtained at yearly exams and physicals to screen for pre-cancerous cells, only using more invasive preventative care when necessary. A cell culture system in which cells display two-fold increase in population doublings before senescence without tumorigenesis has been described (Page et al., 2009 Cloning and Stem Cells 11, 417-426). By altering the conditions under which the cells are grown, cellular lifespan was increased more than twofold (Page et al., 2009 Cloning and Stem Cells, 11:417-426; Page et al., 2011 Tissue Engineering Part A, 17:2629-2640). Increases in cellular lifespan are relevant for the identification and characterization of biomarkers during the transformation from a healthy cell to a pre-cancer cell.

This change in phenotype has been termed extended lifespan (ELS) or induced regeneration competence (iRC), which terms are used interchangeably herein. ELS (also known as iRC) cells are used herein as a tool to characterize an early marker of increased cellular lifespan, offering potential targets for diagnostic tests. Specifically, as described in detail below, ELS cells demonstrate significant up-regulation of the arginine methyltransferase PRMT8 compared to control cells. Aberrant PRMT expression plays a role in various disease states and certain PRMT protein variants are used as prognostic markers of lung and bladder cancers (Zakrzewicz et al., 2012 International Journal of Molecular Sciences, 13:12383-12400; Yoshimatsu et al., 2011 International Journal of Cancer, 128:562-573; Mathioudaki et al., 2008 British Journal of Cancer, 99:2094-2099; and Goulet et al., 2007 Journal of Biological Chemistry, 282:33009-33021). Mutations in PRMT have been identified in skin, ovarian, and colorectal cancers (Yang Y and Bedford Mont., 2013 Nature Reviews Cancer, 13:37-50).

Of relevance are deep sequencing results of cancer genomes that reveal PRMT8 to be the most mutated PRMT family member, having 15 coding region mutations out of the 106 genomes tested (Yang Y and Bedford Mont., 2013 Nature Reviews Cancer, 13:37-50). In contrast, PRMT8 up-regulation in ELS cells is accompanied by increased cellular lifespan in a non-tumorigenic system. Described herein is the development of a prognostic PCR test with pre-cancerous screening capabilities based on up-regulation of PRMT8. Thus, described herein is a greater understanding of PRMT8 up-regulation within ELS cells and association with specific pre-cancer and/or cancer cell types.

Regardless of whether cancers arise as a consequence of genetic or epigenetic changes, the factors that control the balance between replicative senescence and cancerous self-renewal are of much interest as potential therapeutic targets. However, prior to the invention described herein, the molecular mechanisms that regulate this perfect balance were not well understood. As described herein, to better study this regulatory mechanism, an in vitro model system was developed which allows for increase in telomerase reverse transcriptase (TERT) levels leading to increased proliferative potential of the cells and increased time to senescence, while at the same time the cells remain non-tumorigenic when injected into severe combined immunodeficiency (SCID) mice (Page et al., 2009 Cloning and Stem Cells, 11:417-426), leading to the term extended lifespan (ELS) cells. This phenotype is also accompanied by induction of regeneration competence as demonstrated by significant reduction of collagen deposition in a mouse skeletal wound (Page et al., 2011 Tissue Engineering Part A, 17:2629-2640), leading to the term induced regeneration competent (iRC) cells to describe the cells' regenerative phenotype. As described in detail below, understanding the mechanism of cell plasticity in the context of a defined environment offers molecular tools for designing of regenerative instead of symptomatic treatment strategies.

As such, as described in detail below, it is determined whether PRMT8 is involved in increased proliferation of ELS cells by direct or indirect regulation of TERT expression, as elucidation of this pathway uncovers therapeutic targets for regenerative medicine and cancer research. As described herein, the data shows a 13.3 fold transcriptional increase in PRMT8 in ELS cells displaying nuclear localization.

Prior to the invention described herein, identification of molecular mechanisms that regulate cellular replicative lifespan was needed to better understand the transition between a normal and a neoplastic cell phenotype. As described herein, low oxygen-mediated activity of FGF2 leads to an increase in cellular lifespan and acquisition of regeneration competence in human dermal fibroblasts (iRC cells). Though cells display a more plastic developmental phenotype, they remain non-tumorigenic when injected into SCID mice (Page, et al., 2009 Cloning and Stem Cells, 11: 417-426; Page et al., 2011 Tissue Engineering Part A, 17: 2629-2640) allowing for investigation of mechanisms that regulate increased cellular lifespan in a non-tumorigenic system. As described below, analysis of chromatin modification enzymes by qRT-PCR revealed a 13.3-fold upregulation of the arginine methyltransferase PRMT8 in iRC cells. As described in detail herein, increased protein expression was confirmed in both iRC and human embryonic stem cells—the first demonstration of endogenous human PRMT8 expression. Furthermore, as described herein, iRC cells express a PRMT8 mRNA variant. As described herein, using siRNA-mediated knockdown it was demonstrated that this variant was required for viability proliferation of human dermal fibroblasts and grade IV glioblastomas. Thus, PRMT8 upregulation in a non-tumorigenic system is a diagnostic biomarker and a therapeutic target for cells in pre-cancerous and cancerous states.

### Pharmaceutical Therapeutics

For therapeutic uses, the compositions or agents described herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia, although in certain instances lower amounts will be needed because of the increased specificity of the compound. For example, a therapeutic compound is administered at a dosage that is cytotoxic to a neoplastic cell.

### Formulation of Pharmaceutical Compositions

The administration of a compound or a combination of compounds for the treatment of a neoplasia may be by any suitable means that results in a concentration of the therapeutic that, combined with other components, is effective in ameliorating, reducing, or stabilizing a neoplasia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other cases, this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other aspects, it is envisaged that doses may be in the range of about 5 mg compound/Kg body to about 20 mg compound/Kg body. In other embodiments, the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

Pharmaceutical compositions according to the invention may be formulated to release the active compound substantially immediately upon administration or at any predetermined time or time period after administration. The latter types of compositions are generally known as controlled release formulations.

### Kits or Pharmaceutical Systems

The present compositions may be assembled into kits or pharmaceutical systems for use in ameliorating a neoplasia. Kits or pharmaceutical systems according to this aspect of the invention comprise a carrier means, such as a box, carton, tube or the like, having in close confinement therein one or more container means, such as vials, tubes, ampoules, or bottles. The kits or pharmaceutical systems of the invention may also comprise associated instructions for using the agents of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLE 1

### Regeneration Competence Accompanies Increased Expression of Arginine Methyltransferase PRMT8 in Human Adult Fibroblasts

Identification of therapeutically relevant molecules is necessary for the advancement of non-viral reprogramming of human cells for regenerative medicine. Described herein is a non-viral model system that transforms primary human dermal fibroblasts into cells with induced regeneration competence (ELS). As described in detail below, low oxygen-mediated effects of fibroblast growth factor (FGF2) lead to an increased cellular lifespan with a two fold increase in population doublings before senescence, remaining non-tumorigenic when injected into SCID mice while maintaining regeneration competence (Page et al., 2009 Cloning and Stem Cells, 11:417-426; Page et al., 2011 Tissue Engineering Part A, 17:2629-2640). This system allows for the examination of molecules that participate in increased cellular lifespan in a non-tumorigenic system. Described herein is the identification of unique molecules that contribute to the ELS phenotype with the goal to design therapeutics that target diseases associated with aging, wound healing, and tumor formation. Analysis of 84 chromatin modification enzymes by quantitative real-time polymerase chain reaction (qRT-PCR) revealed 13.3-fold upregulation of the arginine methyltransferase, PRMT8, in ELS cells. Increased protein expression was confirmed in both ELS and human embryonic stem cells—the first demonstration of endogenous human PRMT8 expression. Also described herein is the regulation of arginine methyltransferases and the functions of endogenous PRMT8 in human cells.

Corruption of the pathway that maintains cellular senescence is associated with approximately 90% of cancers in humans (Kyo et al., 2008 Cancer Science, 99(8):1528-1538). Identification of molecules that initiate dysregulation of this pathway can be exploited for the development of cancer therapeutics. Major advancements in personalized medicine were made when terminally differentiated cells were reprogrammed into induced pluripotent stem cells (iPSCs). However, translation of this methodology for personalized medicine applications is handicapped by viral addition of reprogramming factors, low reprogramming efficiency, and tumorigenesis.

Described herein is a non-viral cell phenotype from primary human dermal fibroblasts, extended lifespan (ELS) cells. ELS cells are derived by exogenous addition of human fibroblast growth factor FGF2 and reduced oxygen concentration (2%). FGF2 is a critical component of stem cell cultures; it is a mitogen required for maintenance of pluripotency. Reduction in oxygen concentration increases cellular lifespan and regulates epigenetic changes (Jeltsch A, 2013 Trends in Biochemical Sciences, 38(4):172-176). Due to defined changes in culture conditions, ELS cells display increased population doublings, increased time to cellular senescence, and at the same time lack tumor forming capacity when injected into SCID mice (Page et al., 2009 Cloning and Stem Cells, 11:417-426). This unique phenotype allows for the examination of molecular changes that lead to increased cellular lifespan without cancerous self-renewal. At a mouse skeletal wound site, ELS cells engraft and aid in regeneration of skeletal muscle (Page et al., 2011 Tissue Engineering Part A, 17:2629-2640). Thus, culture conditions alone can induce a proregenerative, non-tumorigenic phenotype. Accordingly, a variety of biological questions regarding inhibition of senescence by environmental cues may be examined in ELS cells.

To understand molecular mechanisms that contribute to phenotypic differences between control human dermal fibroblasts and ELS cells, molecules that control epigenetic changes in adult human cells were examined. For example, arginine methyltransferases are emerging regulators of proliferation and differentiation and are established modulators of gene expression (Copeland et al., Nature Reviews Drug Discovery, 2009 8(9):724-732). Aberrant expression of protein arginine methyltransferase (PRMT) family members is associated with cardiovascular and pulmonary diseases and various types of cancers, including lung, bladder, colon, and breast cancers.

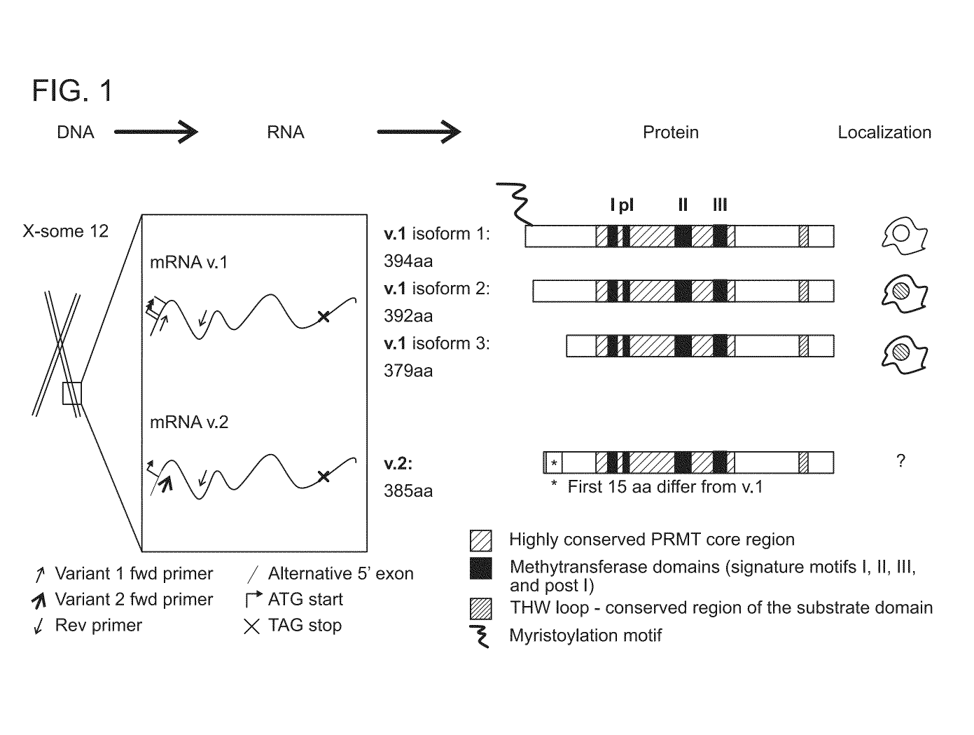

Prior to the invention described herein, little was known regarding the endogenous expression and function of PRMT8. PRMT8 has two mRNA variants transcribed from alternative 5′ exons. Variant 1 has three isoforms with unique N-terminal sequences translated from differing in-frame methionines (FIG. 1). Variant 2 has only been described using genomic sequencing and is thought to have only one protein isoform. Early characterization of PRMT8 variant 1 revealed a myrostylation motif that causes sequestration to the plasma membrane. However, overexpressed PRMT8 variants 2 and 3 display nuclear localization (Kousaka et al., 2009 Neuroscience, 163(4):1146-1157). Study of PRMT8 is guided by the consensus that full-length product is endogenous and expression is restricted to brain tissue. As described herein, if endogenous PRMT8 is nuclear, it challenges the paradigm and becomes more likely that PRMT8, like other family members, has a role in critical cellular processes through chromatin modification or regulation of protein-protein interactions. Prior to the invention described herein, the expression or activity of endogenous PRMT8 in human cells was unknown.

### Materials and Methods

### Cell Culture

Cell culture was performed as described (Page et al., 2009 Cloning and Stem Cells, 11(3):417-426).

### RT-PCR

RNA was prepared using Trizol (Invitrogen). cDNA was synthesized using gScript™ cDNA SuperMix (Quanta Biosciences). PCR was performed using GoTaq (Promega).

qRT-PCR Array Analysis

RNA was prepared using NucleoSpin RNA II kit (Macherey-Nagel). cDNA was synthesized using RT2 First Strand Kit (SABiosciences). Relative quantification was determined using a 7500 Real Time PCR system (Applied Biosystems) measuring SYBR green fluorescence. RT2 Profiler™ PCR Arrays from SABiosciences for chromatin modifying enzymes containing 84 probes were used. Fold change was calculated based on difference in Ct values.

### Western Blotting

Cells were lysed by sonication. Proteins in the lysates were separated using SDS-PAGE and transferred to PVDF membranes. Antibodies used were: PRMT8 (Y. Mori; Novus NBP1-81702) and actin (Sigma A-2006). HRP-conjugated secondary antibodies were used (SantaCruz).

### Immunocytochemistry

Cells were fixed 2% paraformaldehyde and permeabilized with 0.1% Triton X-100 in PBS. Cells were blocked with 5% BSA. Alexafluor-488 labeled secondary antibody (4 μg/mL, Invitrogen) was used. Nuclear counterstaining was added with 0.5 μg/mL Hoechst. Antibodies used were: PRMT8 (Y. Mori; Novus NBP1-87102). Fluorescent images were acquired using confocal microscopy.

### Results

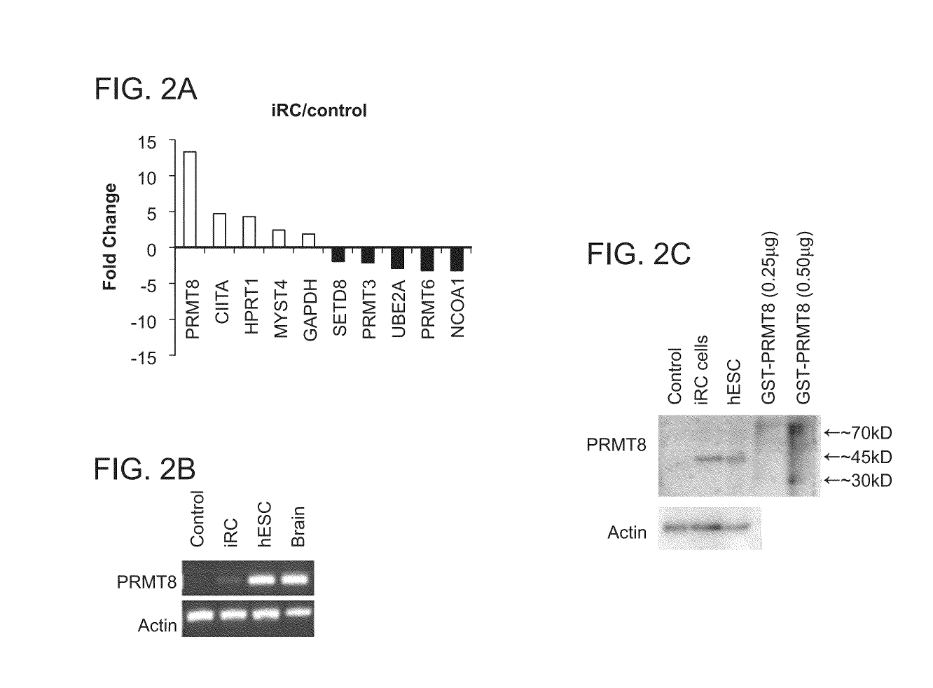

To identify molecular targets that contribute to the ELS phenotype, control cells and ELS cells were harvested at day 7 to perform Human Epigenetic Chromatin Modification Enzyme Arrays (SA Biosciences). Of the 84 genes examined, the most considerable expression change was demonstrated by PRMT8, with 13.3 fold transcriptional increase in ELS cells compared to control cells (FIG. 2A).

Upregulation of PRMT8 transcript in ELS cells was detected using RT-PCR (FIG. 2B) with mouse brain cDNA as a positive control. Of note is the presence of PRMT8 transcript expression in human embryonic stem cells (hESCs).

To determine if upregulation of PRTM8 transcript correlated to upregulation of PRMT8 protein expression, Western blot analysis was performed (FIG. 2C). GST-tagged purified PRMT8 (Y. Mori) was used as a positive control. The 26kD GST tag is responsible for the shift of PRMT8 from 45kD to 7 lkD. These results also demonstrate endogenous PRMT8 protein expression in hESCs for the first time.

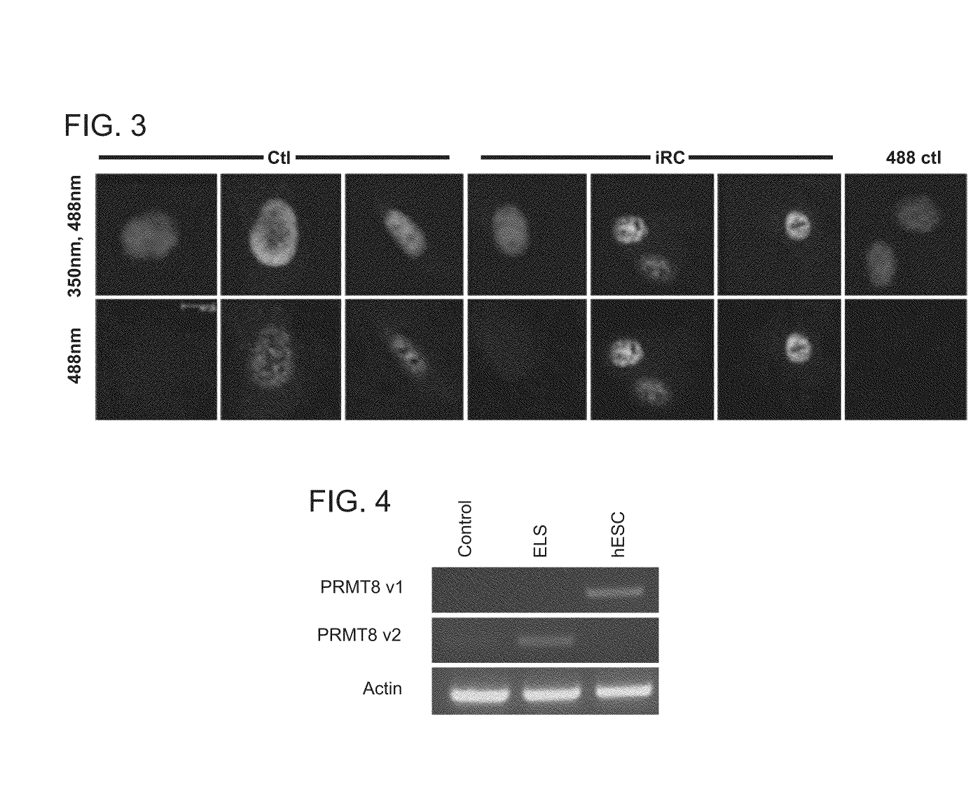

To explore the subcellular localization of endogenous PRMT8 in human cells, immunocytochemistry (ICC) was employed (FIG. 3). PRMT8 localization was restricted almost exclusively to nuclei as PRMT8 expression colocalized with Hoechst nuclear stain.

These data suggest endogenous PRMT8 expressed in ELS cells is likely not the myristoylated full-length isoform, providing similarities between human PRMT8 and reports of endogenous mouse PRMT8 (Kousaka et al., 2009 Neuroscience, 163(4):1146-1157). This work also supports evidence for PRMT8 function outside of the nervous system with a potential role in development. Described in detail below is the functional role of PRMT8 in relation to increased lifespan of ELS cells using its overexpression and knockdown.

### EXAMPLE 2

### Identification of the PRMT8 Variant Up-Regulated in ELS Cells

As described above, PRMT8 is up-regulated in ELS cells compared to control cells at both the transcript and protein level (FIG. 2B and FIG. 2C). Variant specific expression of PRMT1, the family member most similar to PRMT8, increases during progression of tumor formation in colon cancer (Mathioudaki et al., 2008 British Journal of Cancer, 99: 2094-2099). Because of the importance of PRMT variant expression in disease states, the variant identity of ELS-PRMT8 is determined as described herein. Two mRNA variants of PRMT8 have been described with a third mRNA sequence predicted. Human embryonic stem cells (hESCs) and ELS cells were tested by PCR for mRNA variants by variant specific PCR. After analysis, neither variant 2 is present in ELS cells (FIG. 4). The importance of ELS-PRMT8 variant identification for use as a biomarker is underscored by the prevalence of PRMT8 mutations in skin, ovarian, and colorectal cancers (Yang Y. and Bedford M. T., 2013Nature Reviews Cancer, 13:37-50).

PRMT8 variant identification is carried out in two ways: 1) 5′ Rapid Amplification of cDNA Ends (RACE) to identify the mRNA variant present in ELS cells, and 2) LC/MS to sequence the protein isoform present in ELS cells. A 5′ RACE System (Life Technologies®) would enable the identification of the mRNA variant of PRMT8 present in ELS cells. PRMT8 specific primers are purchased from Integrated DNA Technologies (IDT®). PRMT8 antibody is purchased from Novus Biologicals®. Finally, precast polyacrylamide gels are purchased from BioRad®.

### EXAMPLE 3

### The Role of PRMT8 in ELS Cells on the ELS Phenotype

ELS cells demonstrate significant increase in cellular lifespan: while control fibroblasts undergo 33 population doublings over 59 days, ELS cells undergo 68 population doublings over 76 days (Page et al., 2009 Cloning and Stem Cells, 11:417-426). Cellular senescence is critical for maintaining genomic integrity; corruption of the pathway that maintains cellular senescence is associated with approximately 90% of cancers in humans (Kyo et al., 2008 Cancer Science, 99: 1528-1538). Prior to the invention described herein, there have been no reports addressing the effect of PRMT8 expression on cellular senescence. However, an increasing number of publications are identifying roles for other PRMT family members in senescence regulation. In human fibroblasts, PRMT1 protein levels decrease significantly as cells reach replicative senescence (Lim et al., 2008 Journal of biochemistry, 144:523-529). Factors that regulate loss of PRMT1 over the course of cellular lifespan appear to be critical for cellular senescence. PRMT1 is up-regulated in lung and bladder cancer, where abrogation of PRMT1 suppresses cancer cell growth (Yoshimatsu et al., 2011 International Journal of Cancer, 128:562-573). This suggests corruption of the pathway that maintains cellular senescence is accompanied by increased PRMT1 expression.

Prior to the invention described herein, a role for PRMT8 in cellular senescence had not been identified. Described herein is the characterization of the role of PRMT8 on increased lifespan in a non-tumorigenic system.

A protocol is developed that measures differences in telomere length between ELS and control cells, a potential indicator for changes in cellular senescence. This optimized method is utilized to determine if PRMT8 overexpression or knockdown affects telomere length and/or telomerase activity. The role of PRMT8 on the ELS phenotype is assessed with both loss-of-function and gain-of-function experiments.

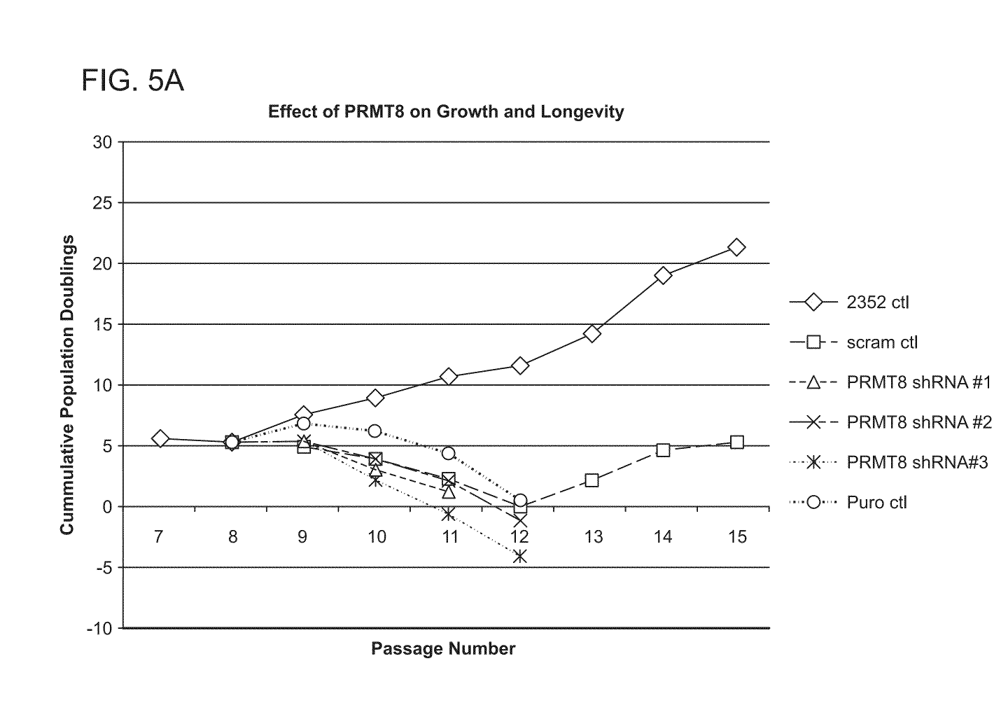

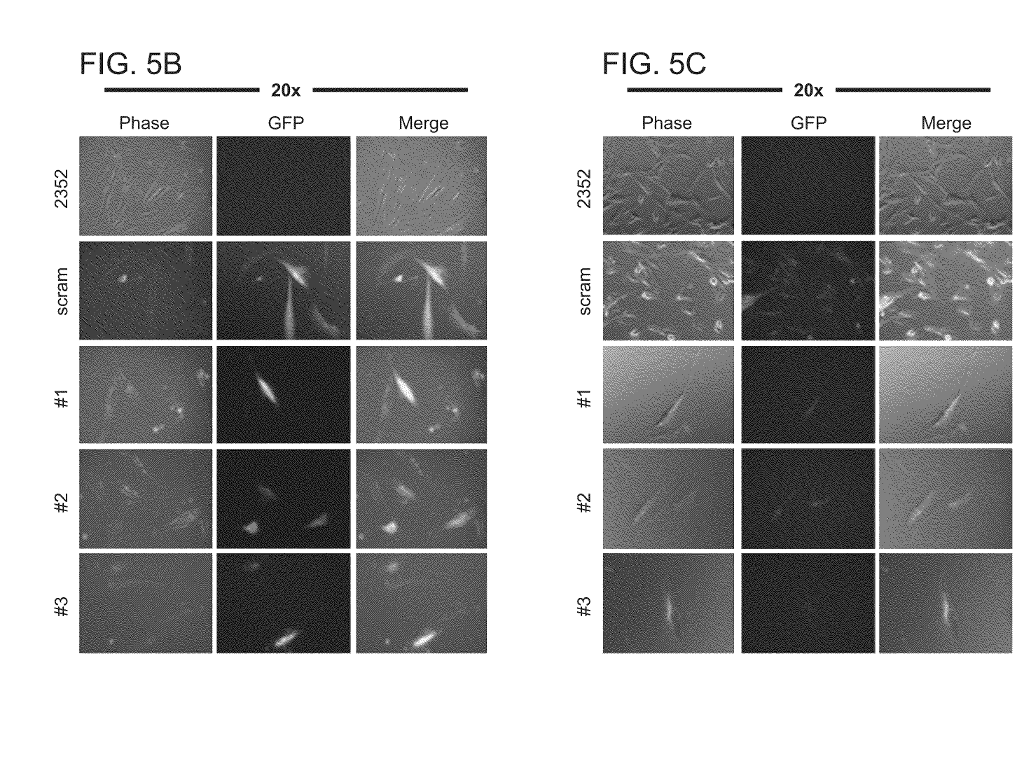

For loss-of-function, lentiviral particles against PRMT8 were purchased from GenTarget Inc. Current experiments are being done to optimize PRMT8 knockdown (FIG. 5A-FIG. 5C). After transduction, primary cells are moved to ELS conditions until senescence. Lentiviral overexpression particles are purchased from transOMIC.

For gain-of-function, the PRMT8 protein sequence from mRNA variant 2 is utilized. Lentiviral particles overexpressing ELS-PRMT8 with a C-terminal GFP-tag and Puromycin resistance are developed (transOMIC). PRMT8 is overexpressed in primary human dermal fibroblasts and overexpression is confirmed with Western blotting. To maintain stable overexpression PRMT8 cell lines, Puromycin selection is used to select for PRMT8 integration. After transfection, primary cells are kept in control conditions until senescence.

As a readout for the ELS phenotype, population doublings and time to cellular senescence is measured in overexpression and knockdown PRMT8 cells. Cells are seeded at a density of 16,000 cells per well of a 24 well plate at each passage. Cultures are maintained in appropriate conditions (either control or ELS) until cells senesce. Senescence is determined as the first calculation of negative population doublings and is confirmed with flow cytometry analysis of senescence associated β-galactosidase, the most widely used biomarker for senescent cells. Population doublings are calculated as log2 (final cell count/initial cell count).

### EXAMPLE 4

### PRMT8 Expression Panel of Cancer Cell Lines

A prognostic test will require correlation of ELS-PRMT8 up-regulation with specific types of pre-cancer and cancer cell types. First, literature is reviewed for up-regulation of PRMT8 in various pre-cancer and cancer type(s). Second, PRMT8 up-regulation is examined in cell lines associated with identified pre-cancer and cancer types(s) by RT-PCR. Finally, various primary tissue types from identified pre-cancers and cancers are examined for up-regulation of ELS-PRMT8 by RT-PCR.

To obtain preliminary data regarding the up-regulation of PRMT8 transcript and its potential association with specific types of pre-cancers and cancers, NCBI and COSMIC (Catalog of Somatic Mutations in Cancer) databases, which curate published gene expression profiles, are reviewed. This provides an inexpensive way to rule out a variety of different cell types from the analysis based on previous experimentation.

When at least one viable cell type is targeted based on previously published data, cell lines corresponding to that specific pre-cancer/cancer type are obtained and tested for ELS-PRMT8 up-regulation with RT-PCR. Focus is placed on cell types that can be obtained using non-invasive methods typically performed during routine physicals, such as blood, stool, or urine collection.

For cell types that demonstrate up-regulation of ELS-PRMT8, a larger sample pool is obtained to determine if the ELS-PRMT8 up-regulation is a common molecular mark of that pre-cancer/cancer type. Samples are obtained from BioServe, a tissue repository of more than 600,000 primary samples from more than 120,000 patients. PRMT8 is considered a biomarker for any per-cancer or cancer type that demonstrates increased PRMT8 expression for a significant number of samples tested compared to patient matched control tissue.

### EXAMPLE 5

### Arginine methyltransferase 8 Isoform is Essential for Cell Viability Proliferation

As described above, aberrant arginine methyltransferase expression is correlated to various cancers. As described in detail below, culture conditions that increase lifespan without tumorigenesis induce expression of a variant of arginine methyltransferase, PRMT8. As described below, this PRMT8 variant is required for cell proliferation. Indeed, molecules that regulate the balance between senescence and unregulated proliferation (e.g., PRMT8) may be indicative of early pre-cancer cells.

### Materials and Methods

The following materials and methods were utilized in this example.

### Cell Culture

The adult human fibroblast line CRL-2352 was obtained from American Tissue Culture Collection (ATCC; Manassas, Va.) at passage 2. The foreskin fibroblast line CRL-2097 was obtained from ATCC. The adult human fibroblast line CT-1005 was obtained from a panniculectomy at UMass Medical (Worcester, Mass.) through their tissue distribution program. Cells were cultured in medium consisting of DMEM: Ham's F12 (50:50; MediaTech) with 10% Fetal Clone III (HyClone). The DMEM (without L-Gln or phenol red) was supplemented with 4 mM fresh L-Gln (MediaTech, Manassas, Va.) prior to use. Cultures were carried out in a 37° C. incubator in a humidified environment of 5% CO₂ and either 19% or 2% 0₂ depending on experimental requirement. All cultures were processed for analyses on day 7. When used, media was supplemented with human recombinant FGF2 (PeproTech) at 4 ng/mL. Human embryonic stem cells—hESCs (W09; WiCell, Madison, Wis.) were cultured on mytomycin C-treated mouse embryonic fibroblasts seeded onto 0.1% gelatin coated six-well plates using 80% Knockout™ DMEM (Invitrogen), 20% Knockout™ serum replacement supplemented with 2.0 mM L-Gln, 0.055 mM 2-mercaptoethanol, and 4.0 ng/mL FGF2, as recommended by the supplier. Glioblastomas (U87MG; ATCC) were cultured in medium consisting of DMEM: Ham's F12 (50:50; MediaTech) with 10% Fetal Clone III (HyClone).

### RT-PCR

RNA was prepared by Trizol (Invitrogen, Inc.) according to the manufacturer's instructions and quantified by spectrophotometry (NanoDrop 2000). One microgram of total RNA was used to perform first strand cDNA synthesis using gScript™ cDNA SuperMix (Quanta Biosciences™). Mouse brain RNA was a generous gift from RXi Pharmaceuticals. For RT-PCR, 50 ng first-strand cDNA was used as a template for each reaction. PCR was performed using 12.5 μL GoTaq (Promega) and 0.2 mM each of forward and reverse primers for PRMT1, PRMT8, PRMT8 variant 1, PRMT8 variant 2, GFP, and actin (Table 2). PCR products from the primary round of amplification were diluted 1:100 with Tris EDTA and the diluted primary PCR product was used as product for the second round of amplification of PRMT8 variant 2 by nested PCR. Amplification products were resolved on 2% agarose gels containing 0.5 μg/mL ethidium bromide in lx TAE buffer and photographed using a BioRad Gel Doc XR System.

qRT-PCR Array Analysis

RNA was prepared using NucleoSpin RNA II kit (Macherey-Nagel) according to the manufacturer's instructions and quantified by spectrophotometry (NanoDrop 2000). Two micrograms of total RNA was used to perform first strand cDNA synthesis using RT2 First Strand Kit (SABiosciences) as recommended by the supplier. Relative quantification was determined using a 7500 Real Time PCR system (Applied Biosystems, Bedford, Mass.) measuring SYBR green fluorescence (RT2 SYBR® Green/ROX qPCR Master Mix, SABiosciences). RT2 Profiler™ PCR Arrays from SABiosciences for chromatin modifying enzymes containing 84 probes were used to identify genes with altered expression in the presence of FGF2 and when oxygen levels were reduced. Analysis was performed by SABiosciences RT2 Profiler PCR Array Data Analysis Template v3.3. Fold change was calculated based on difference in Ct values.

### Cloning

PRMT8 was amplified using RT-PCR described above. The PCR product was resolved on a 2% agarose gel and the 205bp band was excised and cleaned using a NucleoSpin Gel and PCR Clean-up column (Macherey Nagel) according to the manufacturer's instructions. A Klenow (New England Biolabs) reaction was performed using the entire PCR product. The reaction was incubated at room temperature for 15 minutes then stopped with the addition of 10 μM EDTA, followed by a column clean up (NucleoSpin, Macherey Nagel). 7Ong from the Klenow reaction were treated with T4 kinase (New England Biolabs). The kinase reaction was incubated at 37° C. before cleaning over a column (NucleoSpin, Macherey Nagel). A T4 ligation was performed with 2Ong pLVX-puromycin (Clontech Laboratories, Inc.) and PCR product in /a 1:1 ratio overnight at 4° C. 10 μL of ligated pLVX was then transformed into chemically competent E. coli cells. Transformants were incubated on ice for 30 minutes and heat shocked at 42° C. for 45 seconds before 250 μL S.O.C. media was added. Transformants were incubated at 37° C. for 1 hour with agitation prior to overnight incubation on puromycin-containing agar plates at 37° C. Colonies were picked and plasmids were cultured in 3 mL LB broth containing ampicillin overnight with agitation at 37° C. Minipreps were performed on plasmid cultures using a NucleoSpin Plasmid Kit (Macherey Nagel) according to the manufacturer's instructions. Insertion of the PCR product was confirmed with a double restriction digest using 50Ong DNA, 5 units ClaI (New England Biolabs), and 5 units BamHI (New England Biolabs) prior to sequencing (GeneWiz, Cambridge, Mass.).

5′ Rapid Amplification of cDNA Ends 5′ sequences were determined using a 5′ RACE System for Rapid Amplification of cDNA Ends kit (Invitrogen) according to the manufacturer's instructions. Briefly, cDNA was synthesized using a primer specific to PRMT8 (5′-CGAGACCTCGATTTCACAG (SEQ ID NO: 9)), the sample was purified over a column, and the enzyme terminal deoxynucleotidyl transferase (TdT) was used to add a series of cytosine residues to the 3′ end of the product. Nested PCR was then performed, the products were run on a 1.5% agarose gel, and bands were excised, purified (Macherey Nagel; Nucleospin Extract II), and sequenced (GeneWiz, Cambridge, Mass.). Primer sequences for nested amplification are as follows: primary PCR—forward primer provided by Invitrogen (abridged anchor primer); reverse primer 5′-CTTGGCAGCGAACATGGAAA (SEQ ID NO: 10) (hES), 5′-CACCAGTGGATTTTGAAGTCCTTG (SEQ ID NO: 11) (iRC); nested PCR—forward primer provided by Invitrogen (abridged universal amplification primer); reverse primer 5′-CATCCAGTACCACTTTGTCCT (SEQ ID NO: 12)(hES), 5′-CTGGAAACATAAGCCCTCCAGG (SEQ ID NO: 13) (iRC). Transduction

Custom lentiviral particles were designed and produced by GenTarget Inc. (San Diego, CA) to target PRMT8 for knockdown using shRNA. Particles contained shRNA constructs driven by an H1 promoter with a GFPpuromycin reporter tag driven by an RSV promoter. Human dermal fibroblasts were seeded at 1.6×10⁴ cells per well of a 12 well plate and incubated at 37° C. overnight. Media was removed and 0.4 mL serum-free media was added to each treatment well, followed by lentiviral particles to a multiplicity of infection of 50. Cells were incubated at 37° C. for 6 hours. Six hours post-transduction, 1 mL complete media was added to each well. Cells were imaged every 24 hours for GFP expression and cumulative population doublings were determined via cell counts. Glioblastomas were seeded at 4.0x10⁴ cells per well of a 6 well plate and transduced with lentiviral particles to a multiplicity of infection of 50. Transfection efficiency was monitored by expression of GFP on a Zeiss inverted epifluorescence microscope (Axiovert 200M) using AxioVision software (AxioVs40 V 4.8.2.0, service pack 4.8.2 SP1). All images were obtained with an AxioCam MRm camera using a 20xLD Plan-Neofluar objective (20x/0.4 Ph2 Korr) using identical settings.

Protein Isolation and Western blotting

Total protein was isolated from subconfluent cells with cell lysis buffer (200 mM Tris-HCl; pH 7.5, 750 mM NaCl, 40% glycerol, 0.0626% Trition-X 100, 0.025% Tween-20, 0.1% NP-40), supplemented with compete protease inhibitor cocktail (PIC, Santa Cruz Biotechnology). Lysis was performed using sonication (Misonix XL2000) on power 3 with 5 pulses performed 3 times. Protein concentration was determined using Coomassie (Bradford) Protein Assay Kit (Thermo Scientific). Protein supernatant and 5x loading dye (10% SDS, 40% glycerol, 1% Bromophenol Blue, 31.3% 1M Tris-HCl; pH 6.8, 5% 2-βmercaptoethanol) were mixed in a 5:1 ratio and boiled for 5 minutes. Proteins were separated using 12% SDS-PAGE at indicated concentrations of total protein in the lysate and transferred to PVDF membranes (BioRad Laboratories) using Towbin transfer buffer (25 mM Tris Base, 192 mM glycine, 20% methanol, 0.037% SDS). The membranes were blocked with Tween-Tris-buffered saline (TBST: 25 mM Tris Base, 137 mM NaCl, 2.7 mM KCl, 0.2% Tween-20) and 5% dry milk while shaking at room temperature for 60 minutes. Primary antibodies were incubated with the membrane in TBST and 1% dry milk rotating overnight at 4° C.: antiPRMT8 (Novus NBP1-81702; 1:200) and anti-actin (Sigma A-2066; 1:5000). HRPconjugated secondary antibodies (SantaCruz Biotechnologies) were incubated with the membrane in TBST and 1% dry milk rotating at room temperature for 2 hours. Between and after antibody incubations, membranes were washed 4 times for 10 minutes each with TBST. Chemilluminescence signal was developed by luminol (SantaCruz Biotechnolgies) and luminescence detected using a BioRad Gel Doc XR System. Densitometry was used for quantitation of the signal. Obtained values (N=3) were normalized to actin and means compared using one-tailed T-test. Difference between the means was judged at p<0.05.

### Results

### PRMT8 is Expressed in Human Dermal Fibroblasts

In an effort to understand molecular mechanisms associated with increased lifespan in iRC cells, known epigenetic modulators were examined as potential candidates. To identify possible target genes, expression of 84 chromatin modification enzymes were analyzed by an qRT-PCR array in fibroblasts grown under control and iRC culture conditions (n=1). Expression was normalized to the housekeeping gene that showed the least divergent expression between experimental groups, the ribosomal protein RPL13. Fold change was calculated by comparing ΔCt values between treatment groups (ΔΔCt). FIG. 7A shows the top 5 most up- and down-regulated genes, per the array, in iRC cells compared to cells grown in the absence of exogenous FGF2 and at ambient oxygen. Of the 84 genes examined, the most considerable expression change was observed in protein arginine methyltransferase 8 (PRMT8), represented by a 13.3-fold transcriptional increase in iRC cells compared to control cells. Expression levels of all other arginine methyltransferases remained relatively unchanged between culture conditions (FIG. 7B). Expression of the most recently-described PRMT family member, PRMT9, was not assessed.

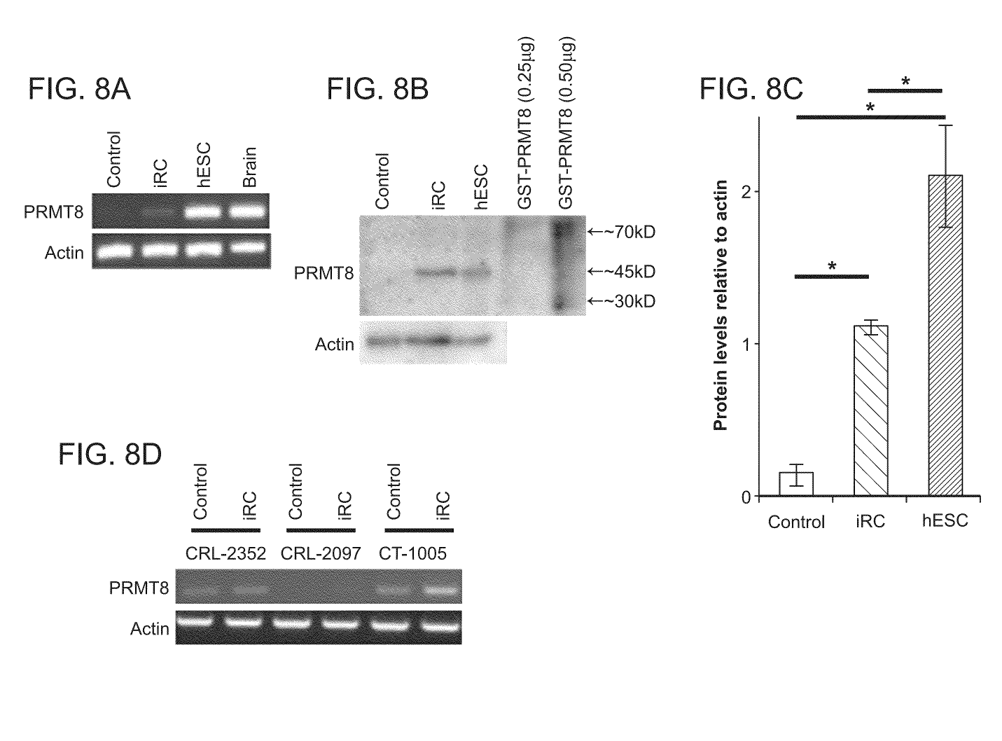

As the array was merely used to identify potential targets for study, PRMT8 was examined as a gene of interest using RT-PCR and Western blotting. Primers were designed to recognize the region of PRMT8 analyzed in the chromatin modification enzyme array. Careful consideration was given during primer design due to high homology between PRMT8 and others within the PRMT family, especially PRMT1 (Lee et al., 2005 Journal of Biological Chemistry, 280: 32890-32896; Hung, C. M. and Li, C. 2004 Gene, 340: 179-187; Lin et al., 2013 PLOS ONE, 8: e55221; Sayegh et al., 2007 Journal of Biological Chemistry, 282: 36444-36453 Kousaka et al., 2009 Neuroscience, 163: 1146-1157). Upregulation of PRMT8 transcript in iRC cells was validated by RT-PCR (FIG. 8A) using mouse brain cDNA as a positive control. Curiously, PRMT8 was also expressed in human embryonic stem cells (hESCs). To determine if upregulation of PRMT8 transcript is accompanied by upregulation of PRMT8 protein expression, Western blot analysis was performed (FIG. 8B). The immunogenic protein was detected at the expected 45kDa in both iRC and hESC cells. The control PRMT8 protein migrated at 7 lkDa, due to its 26kDa GST tag. FIG. 8B is a representative blot; densitometry for 3 replicates can be seen in FIG. 8C. All samples were normalized to actin and analyzed using a onetailed T-test.

As iRC cells are derived from primary human dermal fibroblasts, any sample is subject to individual idiosyncrasies in gene expression. Because of this, PRMT8 expression was analyzed in other primary human dermal fibroblast lines to ensure PRMT8 upregulation is a result of iRC culture conditions and not an artifact of the individual from whom the fibroblasts were derived. Though previous and subsequent work was carried out using CRL-2352s (human adult dermal fibroblasts), other cells, specifically CRL2097s (human foreskin fibroblasts) and CT-1005s (adult female panniculectomy fibroblasts), also demonstrated upregulation of PRMT8 by RT-PCR when grown under iRC culture conditions (FIG. 8D). The significance of low expression in young human tissue (CRL-2097s) compared to high expression in mature human tissue (CRL2352s and CRL-1005s) is not clear. However, even in young tissue expression appears to be inducible by iRC conditions, supporting the idea that regulation of PRMT8 is not dependent on organismal age.

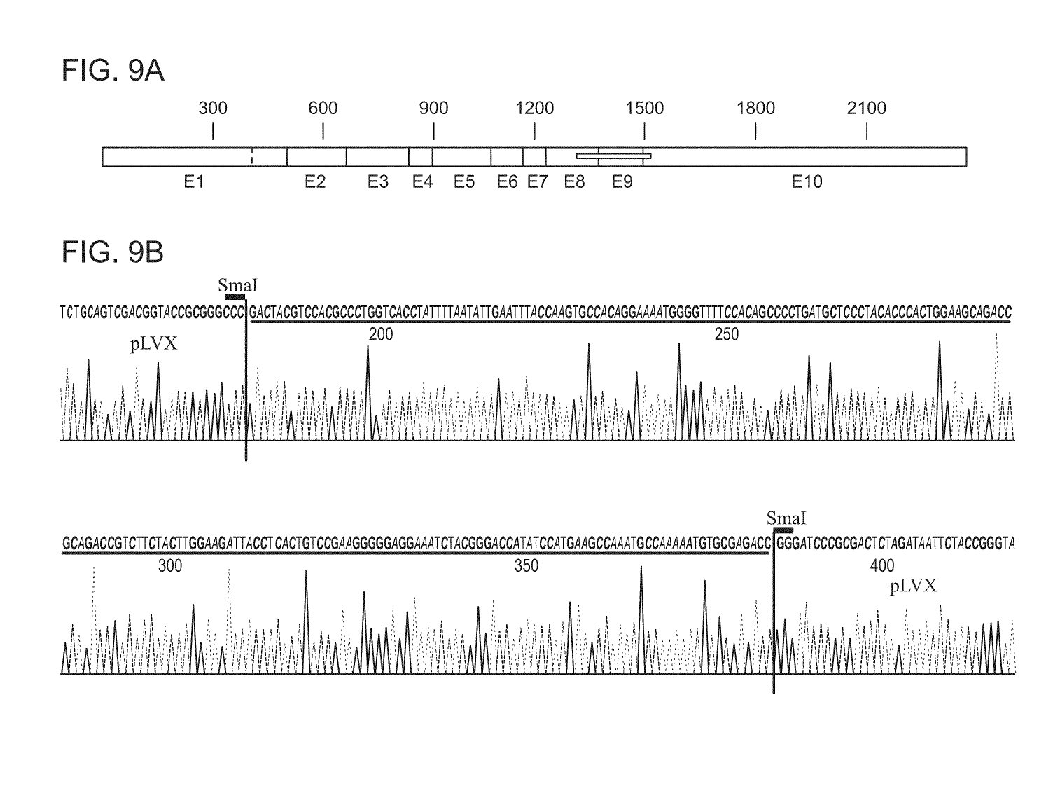

Since consensus opinion relegates expression of PRMT8 strictly to brain tissue (Lee et al., 2005 Endocrine reviews, 26: 147-170; Sayegh et al., 2007 Journal of Biological Chemistry, 282: 36444-36453; Taneda et al., 2007 Brain Research, 1155: 1-9), the transcript detected in iRC cells was sequenced in order to verify the identity of the transcript as PRMT8. PRMT8 was amplified from iRC cells using RT-PCR, the band was excised from the gel, and the fragment was cloned into pLVX using T4 ligase. Positive transformants were sent for sequencing, and the identity of the transcript was verified as that of PRMT8 (FIG. 9).

### Human Dermal Fibroblasts Express a PRMT8 Variant

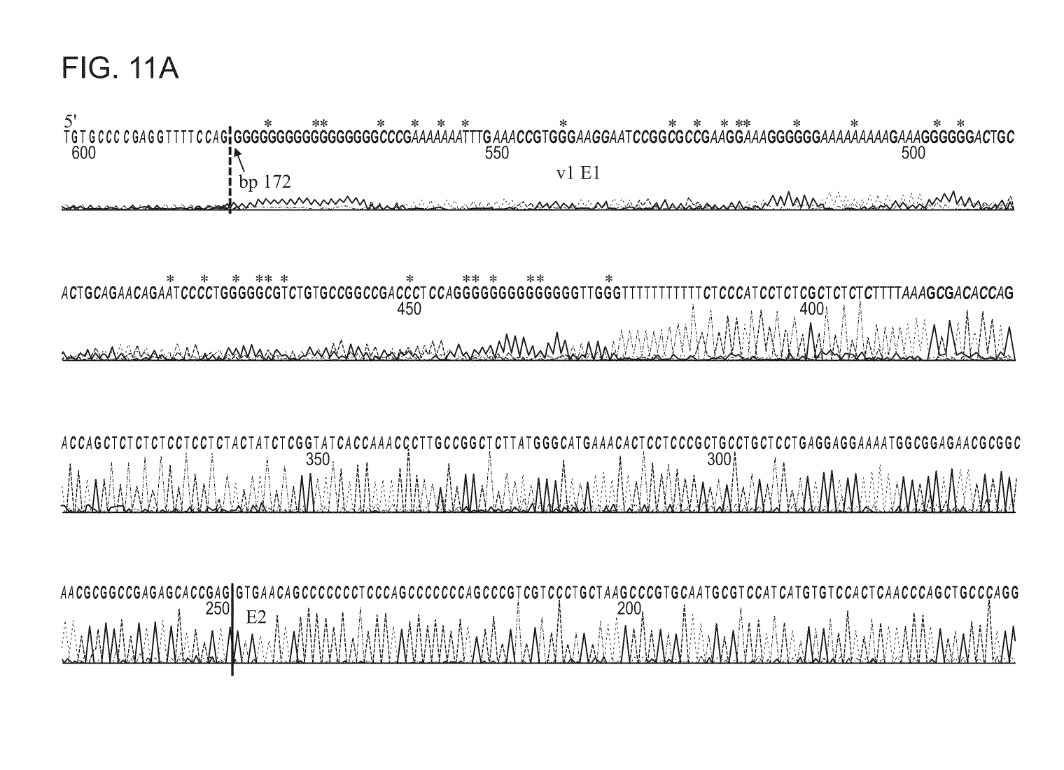

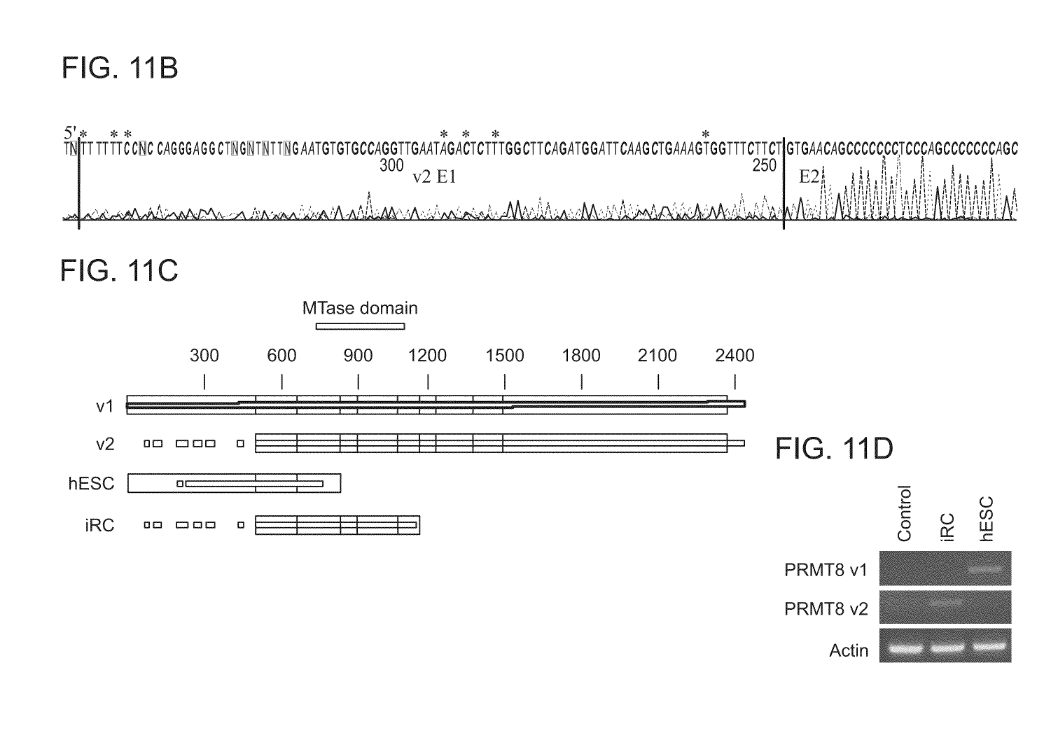

Aberrant PRMT expression plays a role in various disease states, and certain PRMT protein variants are used as prognostic markers for lung and bladder cancers (Yoshimatsu et al., 2011 International Journal of Cancer, 128: 562-573; Zakrzewicz et al., International Journal of Molecular Sciences, 13: 12383-12400; Mathioudaki et al., 2008 British Journal of Cancer, 99: 2094-2099; Goulet et al., 2007 Journal of Biological Chemistry, 282: 33009-33021). As such, it is critical to understand variant and isoform expression of this family of enzymes for development and improvement of diagnostic and therapeutic tools. Through genomic sequencing, a second mRNA variant for PRMT8 was identified (NM_001256536)—one transcribed from an alternate 5′ exon (FIG. 10A). Between mRNA variants 1 and 2, only exon 1 differs, while exons 2 through 10 are identical. FIG. 10B illustratesprimer locations for both mRNA variants used for sequence determination by 5′ Rapid Amplification of cDNA Ends (RACE) and RT-PCR. Also shown are the 4 PRMT8 protein isoforms along with their experimentally determined subcellular localizations. Prior to the invention described herein, only mRNA variant 1 (NM_019854) (incorporated herein by reference) has been studied, along with the 3 protein isoforms translated from that variant (Lee et al., 2005 Journal of Biological Chemistry, 280: 32890-32896; Sayegh et al., 2007 Journal of Biological Chemistry, 282: 36444-36453; Kousaka et al., 2009 Neuroscience, 163: 1146-1157). Isoform 1 harbors a myristoylated residue, conferring plasma membrane localization, while isoforms 2 and 3 are truncated at the N-terminus and lack the myristoylation motif, resulting in nuclear localization (Kousaka et al., 2009 Neuroscience, 163: 1146-1157). 5′ RACE was used to reveal which PRMT8 mRNA variants are expressed in both hESCs and iRC cells (FIG. 11A-FIG. 11B). 5′ RACE with hESC cDNA produced a band that, when sequenced, was identical to exon 1 of PRMT8 mRNA variant 1, beginning with the 172nd nucleotide. 5′ RACE with iRC cell cDNA produced a band that, when sequenced, was identical to exon 1 of PRMT8 mRNA variant 2, beginning with the 1st nucleotide (FIG. 11C). In the National Center for Biotechnology Information (NCBI) database, the sequence for PRMT8 variant 2 was predicted by a combination of genomic DNA and transcript sequences. The cDNA sequence for PRMT8 variant 2 has been curated by NCBI and can be found via the accession number KR014345, incorporated herein by reference.

Forward primers were designed to amplify a region on exon 1 of either variant 1 or variant 2 (FIG. 10A-FIG. 10B) to develop variant-specific PCR so that RT-PCR could be used to test PRMT8 variant expression in other cell types (FIG. 11D). Variant 2 amplification required semi-nested PCR. Sequences for variant-specific primers are presented in Table 2. RT-PCR demonstrated that both control fibroblasts and iRC cells express PRMT8 variant 2 transcript, with control cells expressing low levels, while hESCs express PRMT8 variant 1 transcript, validating the 5′ RACE data.

TABLE 2
DNA primer sequences for RT-PCR

| | | | Amplicon |
|---|---|---|---|
| Primer | Fwd primer (5′ to 3′) | Rev primer (5′ to 3′) | (bp) |
| PRMT1 | CTCTGGTATAAGGCGGTCCC | GCTCATCCCATTAGCCAAGGT | 149 |
| | (SEQ ID NO: 14) | (SEQ ID NO: 19) | |
| PRMT8 | GACTACGTCCACGCCCTGGT | GGTCTCGCACATTTTTGGCATTT | 205 |
| | CACCTATTTTATT | GGCTTCATGG | |
| | (SEQ ID NO: 1) | (SEQ ID NO: 5) | |
| PRMT8 | AAGGAATCCGGAGCAGATG | GGCATAGGAGTCGAAGTAATAA | 458 |
| v1 | AGAAG | TCTCTC | |
| | (SEQ ID NO: 2) | (SEQ ID NO: 6) | |
| PRMT8 | CTGTTTGAATGTGTGCCAGG | GGCATAGGAGTCGAAGTAATAA | 240 |
| v2 | TTG | TCTCTC | |
| | (SEQ ID NO: 3) | (SEQ ID NO: 6) | |
| PRMT8 | TGAATGTGTGCCAGGTTGAA | GGCATAGGAGTCGAAGTAATAA | 235 |
| v2 | TGGAG | TCTCTC | |
| nested | (SEQ ID NO: 4) | (SEQ ID NO: 6) | |
| GFP | AGCTGACCCTGAAGTTCATC | CTGCTTGTCGGCCATGATATAGA | 350 |
| | TG | C | |
| | (SEQ ID NO: 15) | (SEQ ID NO: 18) | |
| Actin | TCTGGCACCACACCTTCTAC | CTTCTCCTTAATGTCACGCACG | 392 |
| | AA | (SEQ ID NO: 19) | |
| | (SEQ ID NO: 16) | | |

### PRMT8 Variant 2 is Critical for Proliferation of Human Dermal Fibroblasts

The iRC phenotype is, in part, characterized by increased cellular lifespan. Because of this, it was next examined whether there is a causal link between increased lifespan and upregulation of PRMT8. PRMT8 was knocked down using custom lentiviral particles containing shRNA constructs designed to target both known mRNA variants of PRMT8—shRNA vector #1 targets PRMT8 within exon 4, shRNA #2 within exon 6, and shRNA #3 within exon 9.

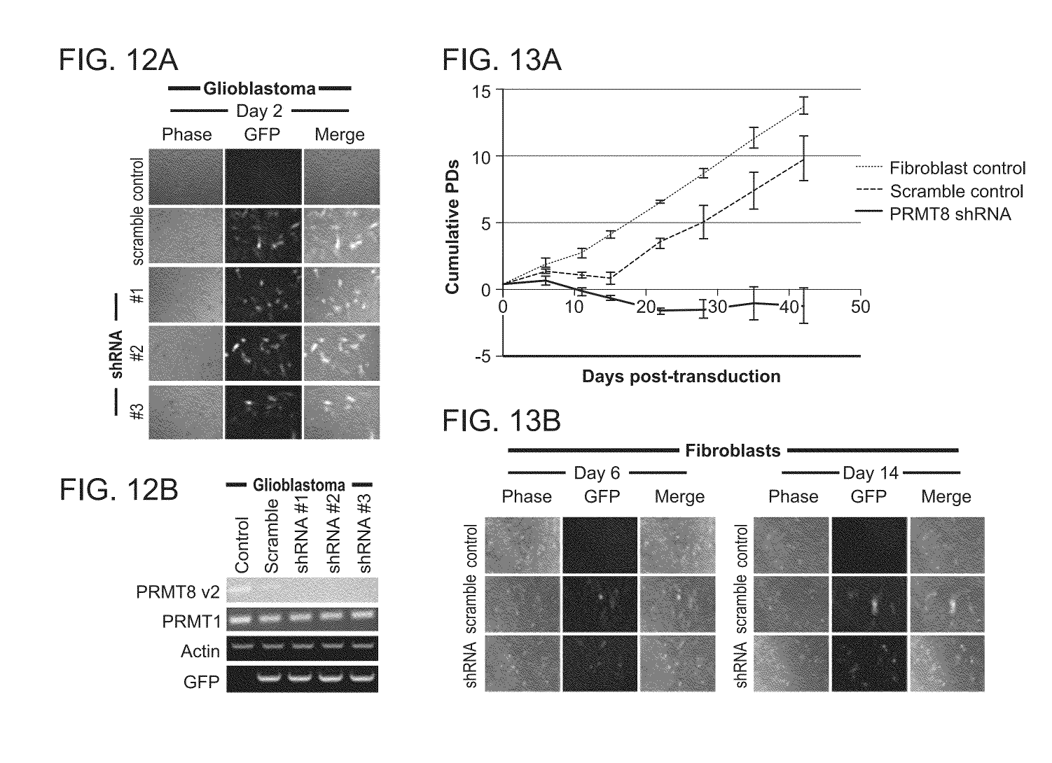

To demonstrate knockdown success and specificity, the glioblastoma line U87MG was transduced with each shRNA construct separately, including the scramble control, and cells were imaged and then harvested 2 days post-transduction for analysis by RT-PCR. Microscopy demonstrated all treatment groups transduced with the scramble control construct, shRNA #1 (ACCACTTGGACAACATCATCAcgagTGATGATGTTGTCCAAGTGGT (SEQ ID NO: 26), shRNA #2 (AGCTTTGTACGTGGTAGCGATcgagATCGCTACCACGTACAAAGCT (SEQ ID NO: 27), and shRNA #3 (GGAAGCAGACCGTCTTCTACTcgagAGTAGAAGACGGTCTGCTTCC (SEQ ID NO: 28), express the GFP reporter, indicating successful transduction in all treatments (FIG. 12A) RT-PCR demonstrated successful PRMT8 knockdown, as control and scramble control treatments express PRMT8 variant 2 transcript, while knockdown treatments do not at detectable levels (FIG. 12B). In each of SEQ ID NOs: 26-28, capital letters to the left comprise the sense strand; lower case letters in the middle comprise the loop; and capital letters to the right comprise the anti-sense strand. Based on this data, shRNA construct #2 was selected for use in experiments to determine the effect of PRMT8 on cellular lifespan. PRMT1 was used to determine knockdown specificity since PRMT8 and PRMT1 are the most homologous members of the PRMT family, sharing over 80% sequence identity (Lee et al., 2005 Journal of Biological Chemistry, 280: 32890-32896; Sayegh et al., 2007 Journal of Biological Chemistry, 282: 36444-36453; Kousaka et al., 2009 Neuroscience, 163: 1146-1157).

Fibroblasts were thawed at passage 7 and transduced at day 0 (FIG. 13A). Puromycin selection pressure was applied for 7 days to all treatment groups except control cells, beginning 3 days after transduction. Control cells reached an average of 13.8 PDs on day 42 post-transduction and control cells receiving scrambled shRNA reached an average of 9.79cumulative PDs after selection recovery on day 42 post-transduction. Reduced viability of cells transduced with scrambled shRNA is likely due to cell type specific effects of transduction on primary cells. It has long been known that primary cells are notoriously difficult to transduce due to low efficiency and excessive cell death—a consequence not observed with immortalized cells (Halbert et al., 1995 Journal of virology, 69: 1473-1479). Cells transduced with PRMT8 shRNA reached an average of −1.26 PDs on day 42 posttransduction with a peak of 0.63 PDs on day 6 post-transduction. Transduction efficiency was monitored by expression of a GFP reporter and GFP fluorescence was imaged weekly throughout the study. FIG. 13B shows representative images from each treatment on day 6 posttransduction and day 14 post-transduction.

### PRMT8 is Critical for Proliferation of Grade IV Glioblastomas

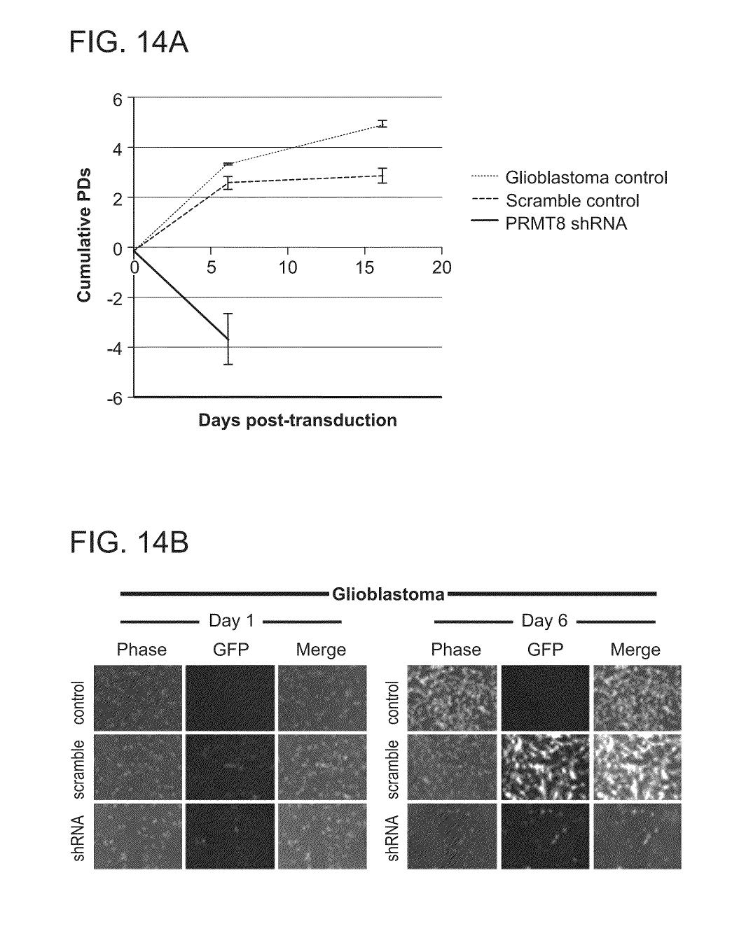

The validity of PRMT8 as a pre-cancer biomarker requires demonstration of the necessity of this gene for proliferation of preneoplastic as well as tumorigenic cells. Accordingly, PRMT8 was next knocked down in glioblastomas to determine whether PRMT8 expression is required for proliferation of this highly aggressive cancer. The glioblastoma line U87MG was transduced at day 0 and puromycin selection pressure was applied for 3 days to all treatment groups except control cells beginning 3 days after transduction (FIG. 14A). Again, three replicates were performed and cumulative PDs were averaged. Control cells reached an average of 4.89 cumulative PDs on day 16 post-transduction. Cells transduced with scramble control shRNA reached an average of 2.83 cumulative PDs on day 16 post-transduction. Cells transduced with PRMT8 shRNA reached an average of -3.73 cumulative PDs on day 6 posttransduction. No data was recorded for PRMT8 shRNA treated cells on day 15 as all cells within the treatment were dead. The experiment was terminated after day 16 due to complete death in the PRMT8 shRNA treatment group. Transduction efficiency was monitored with a GFP reporter and GFP expression in all treatments from day 1 and day 6 can be seen in FIG. 14B.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference.

Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of detecting a pre-cancerous or cancerous cell comprising:
obtaining a test sample from a subject having or at risk of having cancer;
determining the expression level of protein arginine methyltransferase 8 (PRMT8) in said test sample;
comparing the expression level of PRMT8 in said test sample with the expression level of PRMT8 in a reference sample; and
detecting a pre-cancerous or cancerous cell if the expression level of PRMT8 in the test sample is elevated as compared to the expression level of PRMT8 in the reference sample.

2. The method of claim 1, wherein the subject is a human.

3. The method of claim 1, further comprising administering a chemotherapeutic agent, radiation therapy, cryotherapy, or hormone therapy, thereby inhibiting tumor cell growth in said subject.

4. The method of claim 3, wherein the chemotherapeutic agent comprises doceaxel, cabazitaxel, mitoxantrone, estramustine, doxorubicin, etoposide, or paclitaxel.

5. The method of claim 1, further comprising administering an anti-neoplastic agent, wherein said anti-neoplastic agent comprises radiotherapy, a cell death-inducing agent, or a proteasome inhibitor, thereby inhibiting tumor cell growth in said subject.

6. The method of claim 1, wherein said test sample comprises ribonucleic acid (RNA).

7. The method of claim 6, wherein the expression level of PRMT8 messanger ribonucleic acid (mRNA) is determined.

8. The method of claim 7, wherein reverse transcription polymerase chain reaction (RT-PCR) is utilized to determine a level of PRMT8 mRNA in said sample.

9. The method of claim 1, wherein said PRMT8 in said test sample comprises a PRMT8 mRNA variant comprising the nucleic acid sequence set forth in SEQ ID NO: 8.

10. The method of claim 1, further comprising administering an inhibitor of PRMT8 to said subject, thereby inhibiting tumor cell growth.

11. The method of claim 10, wherein said inhibitor of PRMT8 comprises a small molecule inhibitor, RNA interfence (RNAi), an antibody, or any combination thereof.

12. The method of claim 1, wherein the reference sample comprises a tissue-matched normal control sample.

13. The method of claim 1, wherein said test sample comprises a plasma sample, a blood sample, or a tissue sample.

14. The method of claim 1, wherein the reference sample is obtained from a healthy normal control subject.

15. The method of claim 1, wherein said method comprises an in vitro method or an in vivo method.

16. An isolated PRMT8 polypeptide variant.

17. The isolated PRMT8 polypeptide variant of claim 16, wherein said isolated PRMT8 polypeptide variant comprises a synthetic isolated PRMT8 polypeptide variant.

18. The isolated PRMT8 polypeptide variant of claim 16, wherein said polypeptide variant comprises an amino acid sequence set forth in SEQ ID NO: 7.

19. An isolated nucleotide sequence encoding the isolated PRMT8 polypeptide variant of claim 16.

20. The isolated nucleotide sequence of claim 19, wherein said isolated nucleic acid sequence comprises a synthetic isolated nucleic acid sequence.

21. The isolated nucleic acid sequence of claim 20, wherein said isolated nucleic acid sequence comprises complementary deoxyribonucleic acid (cDNA).

22. The isolated nucleotide sequence of claim 21, wherein said isolated nucleic acid sequence is immobilized on a solid support.

23. The isolated nucleic acid sequence of claim 22, wherein said isolated nucleic acid sequence is linked to a detectable label.

24. The isolated nucleic acid sequence of claim 23, wherein said detectable label comprises a fluorescent label, a luminescent label, a chemiluminescent label, a radiolabel, a SYBR Green label, or a Cy3-label.

25. A kit for detecting the expression of PRMT8 mRNA comprising a PRMT8-specific primer.

26. The kit of claim 25, wherein the PRMT8-specific primer comprises a nucleic acid sequence selected from the group consisting of SEQ ID NO: 1 and SEQ ID NO: 5, SEQ ID NO: 2 and SEQ ID NO: 6, SEQ ID NO: 3 and SEQ ID NO: 6, and SEQ ID NO: 4 and SEQ ID NO: 6.
